(19) United States
(12) Patent Application Publication
(10) Pub. No.: US 2023/0112985 A1
(43) Pub. Date: Apr. 13, 2023

(54) BREATH DETECTION WITH MOVEMENT COMPENSATION

(71) Applicant: ResMed Asia Pte. Ltd., Singapore (SG)

(72) Inventors: Kyi Thu MAUNG, Singapore (SG); Yong Sern GWEE, Singapore (SG); Leong Kee CHEE, Singapore (SG); Praveen JAGADEESAN, Singapore (SG)

(73) Assignee: ResMed Asia Pte. Ltd., Singapore (SG)

(21) Appl. No.: 17/908,087

(22) PCT Filed: Mar. 26, 2021
(86) PCT No.: PCT/SG2021/050168

§ 371 Date: Aug. 30, 2022

Related U.S. Application Data
(60) Provisional application No. 63/000,813, filed on Mar. 27, 2020.

(51) Int. Cl. A61M 16/00; A61M 16/10; C01B 13/02; B01D 53/047

(52) U.S. Cl. CPC A61M 16/024; A61M 16/101; C01B 13/0259; B01D 53/047; A61M 2016/0027; A61M 2230/42; A61M 2230/63; C01B 2210/0014; C01B 2210/0046; B01D 2256/12; B01D 2257/102; B01D 2259/4533; B01D 2259/40009

(57) ABSTRACT

An oxygen concentration system may comprise a pressure sensor, a movement sensor, and a controller configured to use one or more pressure signals obtained from the pressure sensor and a movement signal obtained from the movement sensor to determine when to release a bolus of oxygen enriched air. In some implementations, the controller may adjust a trigger threshold based on an initial pressure signal obtained from the pressure sensor and the movement signal obtained from the movement sensor. In some implementations, the controller may adjust a pressure signal obtained from the pressure sensor based on the movement signal obtained from the movement sensor. In some implementations, the controller may detect a potential onset of inhalation from a pressure signal obtained from the pressure sensor and determine whether to verify the potential onset of inhalation based on the movement signal obtained from the movement sensor.

### I. CROSS-REFERENCE TO RELATED APPLICATIONS

This application claims priority to U.S. Provisional Application No. 63/000,813, filed on Mar. 27, 2020, which is incorporated herein by reference.

### II. FIELD OF THE TECHNOLOGY

The present technology generally relates to systems and methods for producing oxygen enriched air for treating respiratory disorders. In some implementations, a combination of respiratory data and movement data is used to efficiently provide the oxygen enriched air to a user.

### III. DESCRIPTION OF THE RELATED ART

A. Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Examples of respiratory disorders include respiratory failure, Obesity Hyperventilation Syndrome (OHS), Chronic Obstructive Pulmonary Disease (COPD), Neuromuscular Disease (NMD) and Chest wall disorders.

Respiratory failure is an umbrella term for respiratory disorders in which the lungs are unable to inspire sufficient oxygen or exhale sufficient CO₂ to meet the patient's needs. Respiratory failure may encompass some or all of the following disorders.

A patient with respiratory insufficiency (a form of respiratory failure) may experience abnormal shortness of breath on exercise.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterized by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive. Rapidly progressive disorders are characterized by muscle impairment that worsens over months and results in death within a few years (e.g., Amyotrophic lateral sclerosis (ALS) and Duchenne muscular dystrophy (DMD) in teenagers). Variable or slowly progressive disorders are characterized by muscle impairment that worsens over years and only mildly reduces life expectancy (e.g., Limb girdle, Facioscapulohumeral and Myotonic muscular dystrophy). Symptoms of respiratory failure in NMD include: increasing generalized weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterized by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

B. Respiratory Therapies

Various respiratory therapies, such as Non-invasive ventilation (NIV), Invasive ventilation (IV), and High Flow Therapy (HFT) have been used to treat one or more of the above respiratory disorders.

1. Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube. In some forms, the comfort and effectiveness of these therapies may be improved.

2. Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched air. In one example, High Flow therapy (HFT) is the provision of a continuous, heated, humidified flow of air to an entrance to the airway through an unsealed or open patient interface at a “treatment flow rate” that is held approximately constant throughout the respiratory cycle. The treatment flow rate is nominally set to exceed the patient's peak inspiratory flow rate. HFT has been used to treat OSA, CSR, respiratory failure, COPD, and other respiratory disorders. One mechanism of action is that the high flow rate of air at the airway entrance improves ventilation efficiency by flushing, or washing out, expired CO₂ from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT) or supplemental oxygen therapy. Doctors may prescribe a continuous flow of oxygen enriched air at a specified oxygen concentration (from 21%, the oxygen fraction in ambient air, to 100%) at a specified flow rate (e.g., 1 litre per minute (LPM), 2 LPM, 3 LPM, etc.) to be delivered to the patient's airway.

3. Supplementary Oxygen

For certain patients, oxygen therapy may be combined with a respiratory pressure therapy or HFT by adding supplementary oxygen to the pressurized flow of air. When oxygen is added to respiratory pressure therapy, this is referred to as RPT with supplementary oxygen. When oxygen is added to HFT, the resulting therapy is referred to as HFT with supplementary oxygen.

C. Respiratory Therapy Systems

These respiratory therapies may be provided by a respiratory therapy system or device. Such systems and devices may also be used to screen, diagnose, or monitor a condition without treating it. A respiratory therapy system may comprise an oxygen source, an air circuit, and a patient interface.

1. Oxygen Source

Experts in this field have recognized that exercise for respiratory failure patients provides long term benefits that slow the progression of the disease, improve quality of life and extend patient longevity. Most stationary forms of exercise like tread mills and stationary bicycles, however, are too strenuous for these patients. As a result, the need for mobility has long been recognized. Until recently, this mobility has been facilitated by the use of small compressed oxygen tanks or cylinders mounted on a cart with dolly wheels. The disadvantage of these tanks is that they contain a finite amount of oxygen and are heavy, weighing about 50 pounds when mounted.

Oxygen concentrators have been in use for about 50 years to supply oxygen for respiratory therapy. Oxygen concentrators may implement processes such as vacuum swing adsorption (VSA), pressure swing adsorption (PSA), or vacuum pressure swing adsorption (VPSA). For example, oxygen concentrators, e.g., POCs, may work based on depressurization (e.g., vacuum operation) and/or pressurization (e.g., compressor operation) in a swing adsorption process (e.g., Vacuum Swing Adsorption, Pressure Swing Adsorption or Vacuum Pressure Swing Adsorption, each of which are referred to herein as a “swing adsorption process”). Pressure swing adsorption may involve using one or more compressors to increase gas pressure inside one or more canisters that contains particles of a gas separation adsorbent. Such a canister when containing a mass of gas separation adsorbent such as a layer of gas separation adsorbent may serve as a sieve bed. As the pressure increases, certain molecules in the gas may become adsorbed onto the gas separation adsorbent. Removal of a portion of the gas in the canister under the pressurized conditions allows separation of the non-adsorbed molecules from the adsorbed molecules. The adsorbed molecules may then be desorbed by venting the sieve beds. Further details regarding oxygen concentrators may be found, for example, in U.S. Published Patent Application No. 2009-0065007, published Mar. 12, 2009, and entitled “Oxygen Concentrator Apparatus and Method”, which is incorporated herein by reference.

Ambient air usually includes approximately 78% nitrogen and 21% oxygen with the balance comprised of argon, carbon dioxide, water vapor and other trace gases. If a gas mixture such as air, for example, is passed under pressure through a canister containing a gas separation adsorbent that attracts nitrogen more strongly than it does oxygen, part or all of the nitrogen will stay in the canister, and the gas coming out of the canister will be enriched in oxygen. When the sieve bed reaches the end of its capacity to adsorb nitrogen, the adsorbed nitrogen may be desorbed by venting. The sieve bed is then ready for another cycle of producing oxygen enriched air. By alternating pressurization cycles of the canisters in a two-canister system, one canister can be separating oxygen while the other canister is being vented (resulting in a near-continuous separation of oxygen from the air). In this manner, oxygen enriched air can be accumulated, such as in a storage container or other pressurizable vessel or conduit coupled to the canisters, for a variety of uses including providing supplemental oxygen to users.

Vacuum swing adsorption (VSA) provides an alternative gas separation technique. VSA typically draws the gas through the separation process of the sieve beds using a vacuum such as a compressor configured to create a vacuum within the sieve beds. Vacuum Pressure Swing Adsorption (VPSA) may be understood to be a hybrid system using a combined vacuum and pressurization technique. For example, a VPSA system may pressurize the sieve beds for the separation process and also apply a vacuum for depressurizing the sieve beds.

Traditional oxygen concentrators have been bulky and heavy making ordinary ambulatory activities with them difficult and impractical. Recently, companies that manufacture large stationary oxygen concentrators began developing portable oxygen concentrators (POCs). The advantage of POCs is that they can produce a theoretically endless supply of oxygen enriched air and provide mobility for patients (users). In order to make these devices small for mobility, the various systems necessary for the production of oxygen enriched air are condensed. POCs seek to utilize their produced oxygen enriched air as efficiently as possible, in order to minimize weight, size, and power consumption. This may be achieved by delivering the oxygen as series of pulses or boluses, each bolus timed to coincide with the start of inspiration. This therapy mode is known as pulsed oxygen delivery (POD) or demand mode, in contrast with traditional continuous flow delivery more suited to stationary oxygen concentrators. POD mode may be implemented with a conserver, which is essentially an active valve with a sensor to determine the onset of inhalation.

2. Air Circuit

An air circuit is a conduit or a tube constructed and arranged to allow, in use, a flow of air to travel between two components of a respiratory therapy system such as the oxygen source and the patient interface. In some cases, there may be separate limbs of the air circuit for inhalation and exhalation. In other cases, a single limb air circuit is used for both inhalation and exhalation.

3. Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal (e.g., with a region of the patient's face), to facilitate the delivery of gas at a pressure at sufficient variance with ambient pressure to effect therapy (e.g., at a positive pressure of about 10 cmH₂O relative to ambient pressure). For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

### IV. SUMMARY OF THE TECHNOLOGY

Example methods and apparatus of the present technology may involve control of a respiratory therapy system. For example, in some implementations, at least one movement sensor, such as an accelerometer, may be included in a respiratory therapy system to compensate for noise created by the movement of a user. In some such implementations, data from the at least one movement sensor may be used to supplement the detection of the user's breaths by identifying potentially noisy data from at least one other sensor, such a flow rate sensor and/or a pressure sensor. By identifying potentially noisy data from the at least one other sensor, the occurrence of false breath detections may be minimized or avoided, and overall power consumption of the respiratory therapy system may be reduced.

One aspect of the present disclosure relates to an oxygen concentration system comprising a pressure sensor, a movement sensor, and one or more processors. The pressure sensor is pneumatically coupled to a delivery conduit for providing a user with oxygen enriched air and configured to generate pressure signals. The movement sensor is configured to generate a movement signal. The one or more processors are communicatively coupled to the pressure sensor and the movement sensor and configured to adjust a trigger threshold based on an initial pressure signal obtained from the pressure sensor and the movement signal obtained from the movement sensor and compare the adjusted trigger threshold with a subsequent pressure signal obtained from the pressure sensor to determine when to provide the user with a bolus of oxygen enriched air through the conduit.

In some implementations, the one or more processors are further configured to maintain the trigger threshold when a magnitude or a frequency of the movement signal is greater than a predetermined threshold.

In some implementations, adjusting the trigger threshold based on the initial pressure signal and the movement signal comprises generating an activity signal, increasing a magnitude of the trigger threshold when a window of the activity signal indicates an increase in the user's activity, and decreasing a magnitude of the trigger threshold when the window of the activity signal indicates a decrease in the user's activity.

In some implementations, generating the activity signal comprises deriving at least one breathing parameter from the initial pressure signal, deriving at least one movement parameter from the movement signal, and combining the at least one breathing parameter and the at least one movement parameter to generate the activity signal. In some implementations, the at least one breathing parameter is a breathing rate of the user, and the at least one movement parameter is a number of steps taken per unit of time by the user.

In some implementations, generating the activity signal comprises generating a non-respiratory signal from the initial pressure signal and scaling the non-respiratory signal based on the movement signal to generate the activity signal. In some implementations, a filter is applied to the initial pressure signal to generate the non-respiratory signal.

In some implementations, a length of the window is fixed. In some implementations, adjusting the trigger threshold based on the initial pressure signal and the movement signal further comprises adjusting a length of the window based on the movement signal. In some implementations, adjusting the length of the window based on the movement signal comprises shortening the length of the window when a magnitude or a frequency of the movement signal is greater than a predetermined threshold.

In some implementations, the movement sensor comprises an accelerometer coupled to the delivery conduit. In some implementations, the movement sensor comprises a strain gauge coupled to the delivery conduit.

In some implementations, the oxygen concentration system further comprises a compression system configured to generate a pressurized stream of ambient air and a canister system comprising a canister containing a gas separation adsorbent, wherein the gas separation adsorbent is configured to separate at least some nitrogen from the pressurized stream of ambient air to produce oxygen enriched air.

Another aspect of the present disclosure relates to an oxygen concentration system comprising a pressure sensor, a movement sensor, and one or more processors. The pressure sensor is pneumatically coupled to a delivery conduit for providing a user with oxygen enriched air and configured to generate pressure signals. The movement sensor is configured to generate a movement signal. The one or more processors are communicatively coupled to the pressure sensor and the movement sensor and configured to adjust a pressure signal obtained from the pressure sensor based on the movement signal obtained from the movement sensor and compare a trigger threshold with the adjusted pressure signal to determine when to provide the user with a bolus of oxygen enriched air through the conduit.

In some implementations, the movement sensor comprises an accelerometer coupled to the delivery conduit. In some implementations, adjusting the pressure signal based on the movement signal comprises analyzing a direction of acceleration derived from the movement signal in relation to an orientation of the pressure sensor. In some implementations, the movement sensor comprises a strain gauge coupled to the delivery conduit, and adjusting the pressure signal based on the movement signal comprises analyzing a measured bending of one or more portions of the delivery conduit. In some implementations, the one or more processors are further configured to adjust the trigger threshold based on the movement signal before the trigger threshold is compared with the adjusted pressure signal.

Yet another aspect of the present disclosure relates to an oxygen concentration system comprising a pressure sensor, a movement sensor, and one or more processors. The pressure sensor is pneumatically coupled to a delivery conduit for providing a user with oxygen enriched air and configured to generate pressure signals. The movement sensor is configured to generate a movement signal. The one or more processors are communicatively coupled to the pressure sensor and the movement sensor and configured to detect a potential onset of inhalation by comparing a trigger threshold with a pressure signal obtained from the pressure sensor, determine whether to verify the potential onset of inhalation based on the movement signal obtained from the movement sensor, and provide the user with a bolus of oxygen enriched air through the conduit if the potential onset of inhalation is verified.

In some implementations, determining whether to verify the potential onset of inhalation based on the movement signal comprises comparing a magnitude of the movement signal to a predetermined threshold. In some implementations, the potential onset of inhalation is verified if the magnitude of the movement signal is less than the predetermined threshold. In some implementations, determining whether to verify the potential onset of inhalation based on the movement signal comprises comparing a frequency of the movement signal to a predetermined threshold. In some implementations, the potential onset of inhalation is verified if the frequency of the movement signal is less than the predetermined threshold. In some implementations, the movement sensor comprises an accelerometer coupled to the delivery conduit.

Yet another aspect of the present disclosure relates to a method of generating a trigger signal for controlling release of a bolus of oxygen enriched gas from an oxygen concentrator that comprises calculating a trigger threshold from an initial pressure signal representing an airway pressure of a user and a movement signal, comparing a subsequent pressure signal representing the airway pressure of the user with the trigger threshold, and generating, based on the comparison, the trigger signal for controlling release of the bolus.

Yet another aspect of the present disclosure relates to a method of generating a trigger signal for controlling release of a bolus of oxygen enriched gas from an oxygen concentrator that comprises adjusting a pressure signal representing an airway pressure of a user based on a movement signal, comparing the adjusted pressure signal with a trigger threshold, and generating, based on the comparison, the trigger signal for controlling release of the bolus.

Yet another aspect of the present disclosure relates to a method of generating a trigger signal for controlling release of a bolus of oxygen enriched gas from an oxygen concentrator that comprises comparing a pressure signal with a trigger threshold to detect a potential onset of inhalation, determining whether to verify the potential onset of inhalation based on a movement signal, and generating, based on the verifying, the trigger signal for controlling release of the bolus.

### V. BRIEF DESCRIPTION OF THE DRAWINGS

Advantages of the present technology will become apparent to those skilled in the art with the benefit of the following detailed description of implementations and upon reference to the accompanying drawings in which similar reference numerals indicate similar components:

FIG. 1A depicts an oxygen concentrator in accordance with one form of the present technology.

FIG. 1B is a schematic diagram of the components of the oxygen concentrator of FIG. 1A.

FIG. 1C is a side view of the main components of the oxygen concentrator of FIG. 1A.

FIG. 1D is a perspective side view of a compression system of the oxygen concentrator of FIG. 1A.

FIG. 1E is a side view of a compression system that includes a heat exchange conduit.

FIG. 1F is a schematic diagram of example outlet components of the oxygen concentrator of FIG. 1A.

FIG. 1G depicts an outlet conduit for the oxygen concentrator of FIG. 1A.

FIG. 1H depicts an alternate outlet conduit for the oxygen concentrator of FIG. 1A.

FIG. 1I is a perspective view of a disassembled canister system for the oxygen concentrator of FIG. 1A.

FIG. 1J is an end view of the canister system of FIG. 1I.

FIG. 1K is an assembled view of the canister system end depicted in FIG. 1J.

FIG. 1L a view of an opposing end of the canister system of FIG. 1I to that depicted in FIGS. 1J and 1K.

FIG. 1M is an assembled view of the canister system end depicted in FIG. 1L.

FIG. 1N depicts an example control panel for the oxygen concentrator of FIG. 1A.

FIG. 1O depicts a connected respiratory therapy system that includes the oxygen concentrator of FIG. 1A.

FIG. 2 is a block diagram of an adaptive triggering system in accordance with one form of the present technology.

FIG. 3A is a modified version of the schematic diagram of FIG. 1F.

FIG. 3B is a modified version of the schematic diagram of FIG. 1F.

FIG. 4A is a modified version of the block diagram of FIG. 2.

FIG. 4B is a modified version of the block diagram of FIG. 2.

FIG. 4C is a modified version of the block diagram of FIG. 2.

FIG. 4D is a modified version of the block diagram of FIG. 2.

### VI. DETAILED DESCRIPTION OF THE IMPLEMENTATIONS

Implementations of the present technology are described in detail with reference to the drawing figures wherein like reference numerals identify similar or identical elements. It is to be understood that the disclosed implementations are merely examples of the disclosure, which may be implemented in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

A. Implementations of Oxygen Concentrators

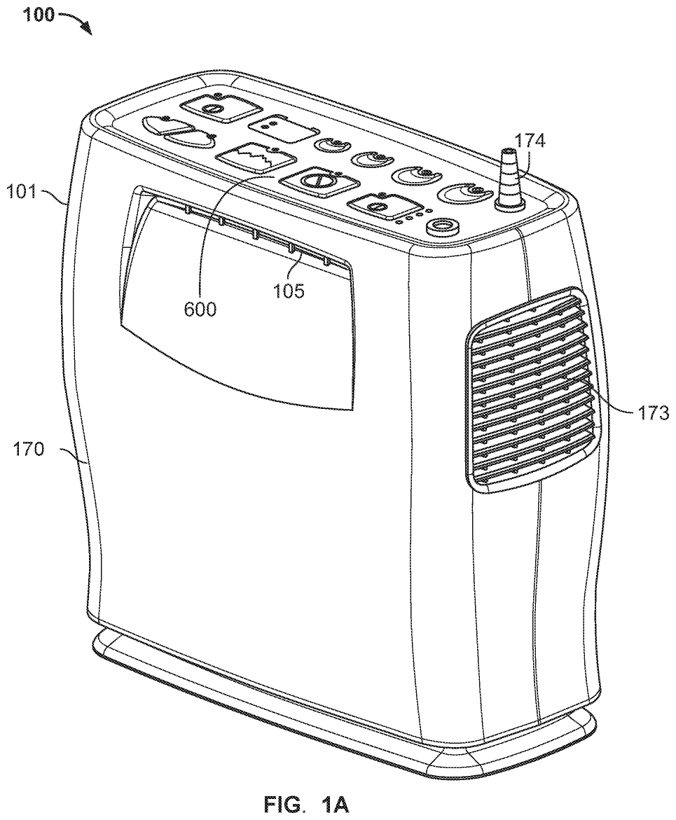

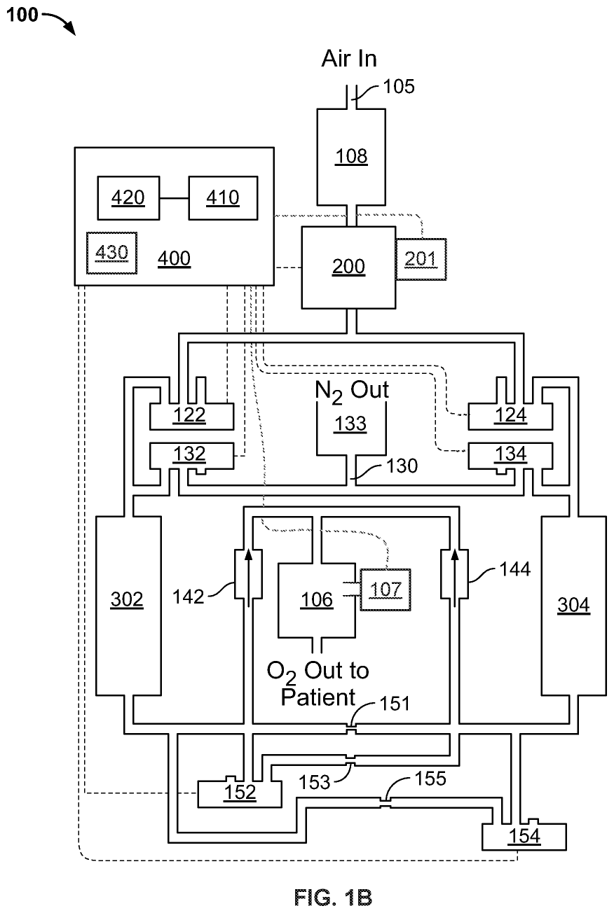

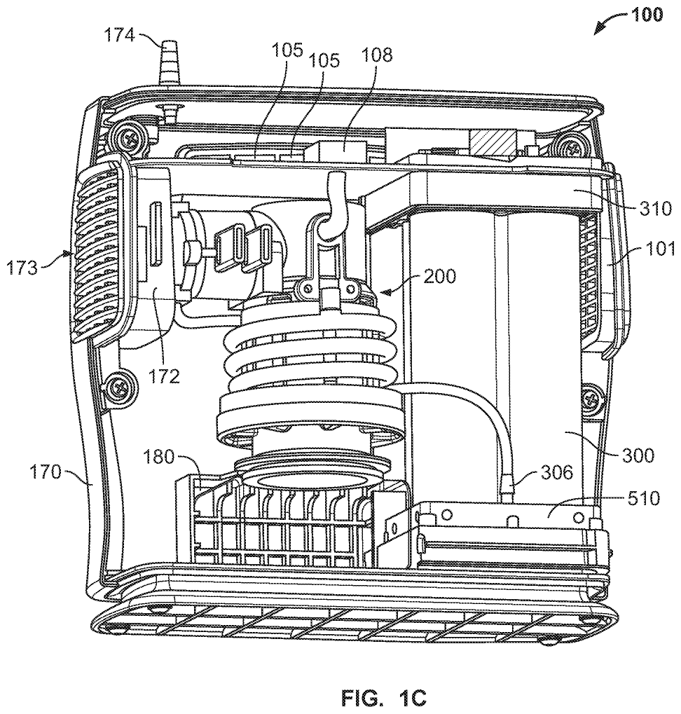

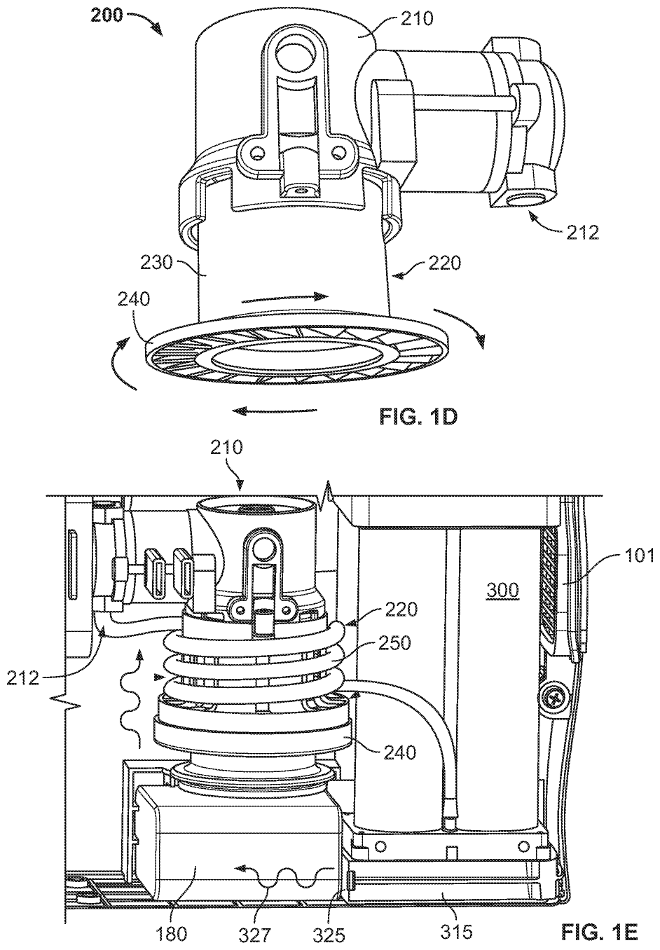

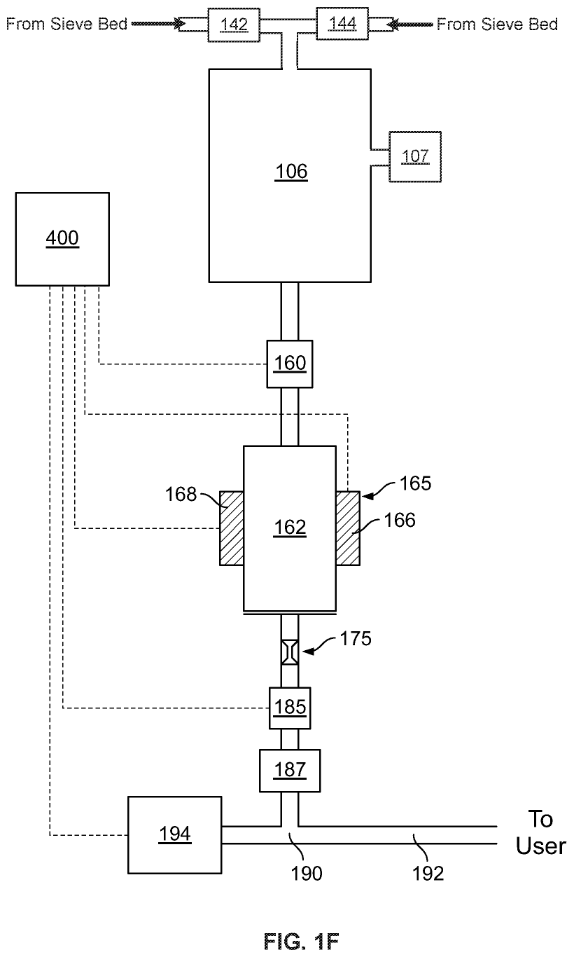

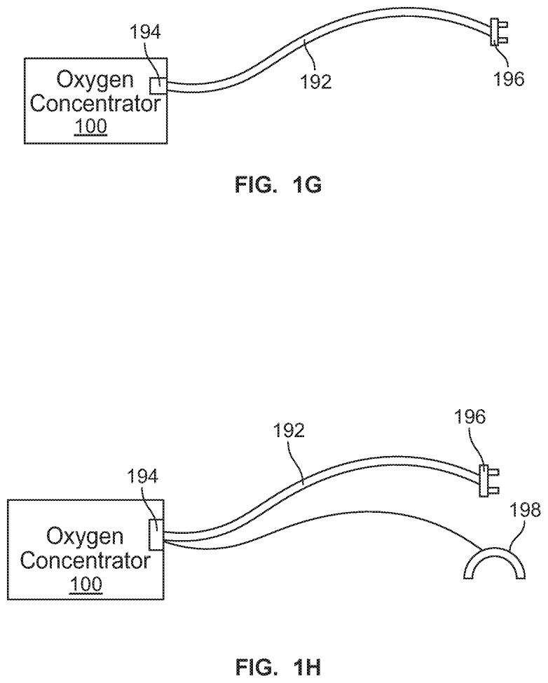

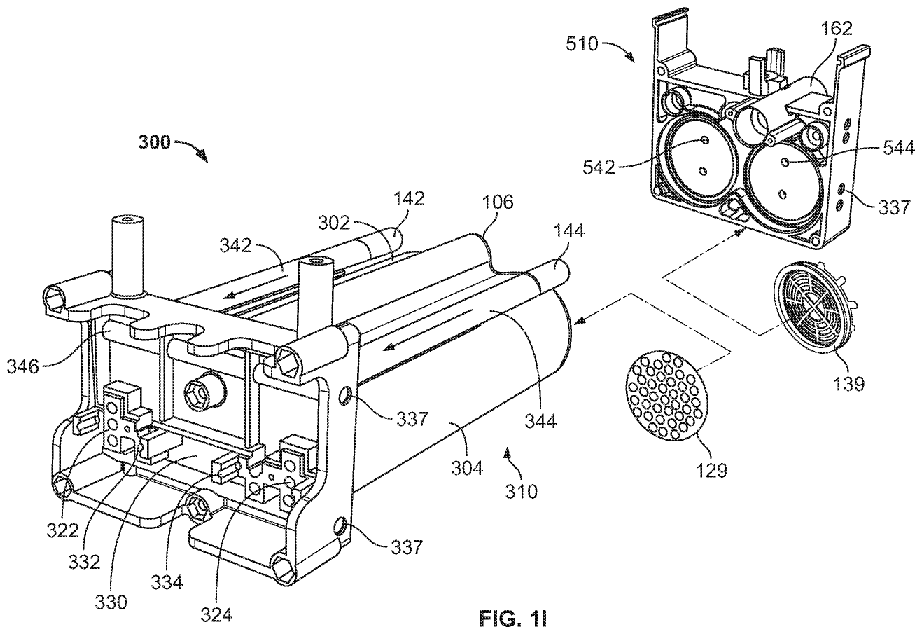

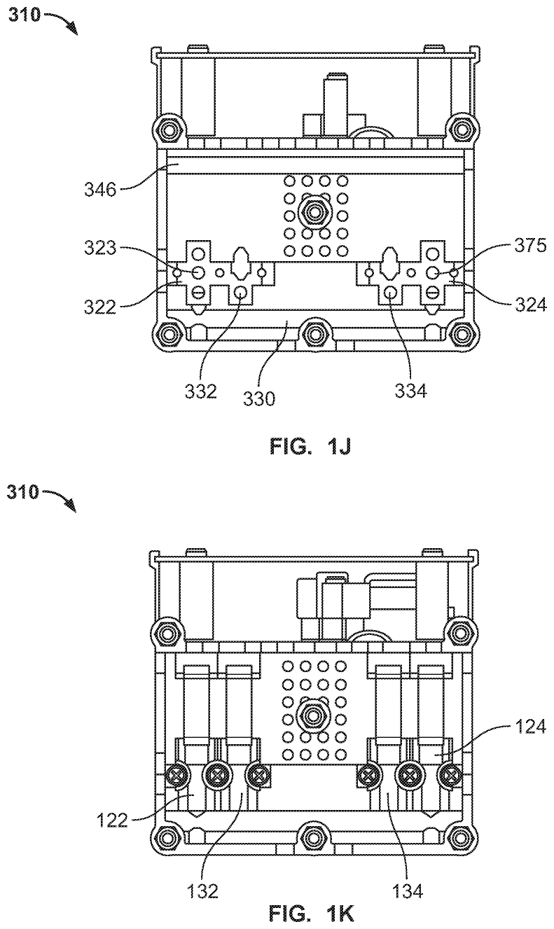

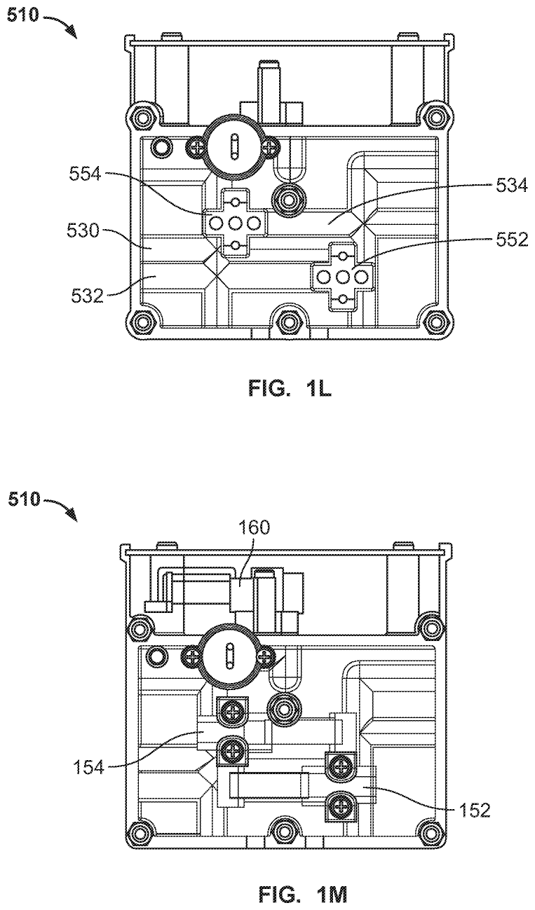

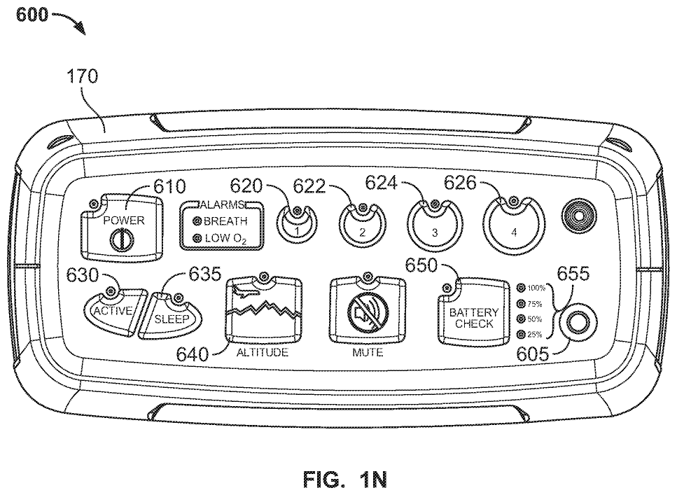

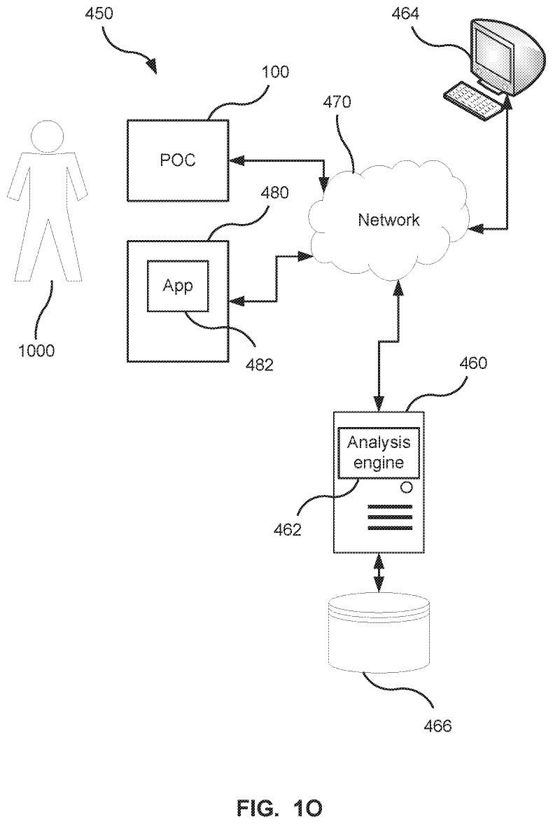

FIGS. 1A-1N illustrate an implementation of an oxygen concentrator 100. As described herein, oxygen concentrator 100 uses pressure swing adsorption (PSA) processes to produce oxygen enriched air. However, in other implementations, oxygen concentrator 100 may be modified such that it uses vacuum swing adsorption (VSA) processes or vacuum pressure swing adsorption (VPSA) processes to produce oxygen enriched air.

1. Outer Housing

FIG. 1A depicts an implementation of an outer housing 170 of an oxygen concentrator 100. In some implementations, outer housing 170 may be comprised of a light-weight plastic. Outer housing 170 includes compression system inlets 105, cooling system passive inlet 101 and outlet 173 at each end of outer housing 170, outlet port 174, and control panel 600. Inlet 101 and outlet 173 allow cooling air to enter the housing, flow through the housing, and exit the interior of housing 170 to aid in cooling of the oxygen concentrator 100. Compression system inlets 105 allow air to enter the compression system. Outlet port 174 is used to attach a conduit to provide oxygen enriched air produced by the oxygen concentrator 100 to a user.

2. Components

FIG. 1B illustrates a schematic diagram of components of an oxygen concentrator 100, according to an implementation. Oxygen concentrator 100 may concentrate oxygen within an air stream to provide oxygen enriched air to a user.

Oxygen concentrator 100 may be a portable oxygen concentrator. For example, oxygen concentrator 100 may have a weight and size that allows the oxygen concentrator to be carried by hand and/or in a carrying case. In one implementation, oxygen concentrator 100 has a weight of less than about 20 pounds, less than about 15 pounds, less than about 10 pounds, or less than about 5 pounds. In an implementation, oxygen concentrator 100 has a volume of less than about 1000 cubic inches, less than about 750 cubic inches, less than about 500 cubic inches, less than about 250 cubic inches, or less than about 200 cubic inches.

Oxygen enriched air may be produced from ambient air by pressurizing ambient air in canisters 302 and 304, which contain a gas separation adsorbent and may be referred to as sieve beds. Gas separation adsorbents useful in an oxygen concentrator are capable of separating at least nitrogen from an air stream to produce oxygen enriched air. Examples of gas separation adsorbents include molecular sieves that are capable of separating nitrogen from an air stream. Examples of adsorbents that may be used in an oxygen concentrator include, but are not limited to, zeolites (natural) or synthetic crystalline aluminosilicates that separate nitrogen from an air stream under elevated pressure. Examples of synthetic crystalline aluminosilicates that may be used include, but are not limited to: OXYSIV adsorbents available from UOP LLC, Des Plaines, IW; SYLOBEAD adsorbents available from W. R. Grace & Co, Columbia, Md.; SILIPORITE adsorbents available from CECA S.A. of Paris, France; ZEOCHEM adsorbents available from Zeochem AG, Uetikon, Switzerland; and AgLiLSX adsorbent available from Air Products and Chemicals, Inc., Allentown, Pa.

As shown in FIG. 1B, air may enter the oxygen concentrator through air inlet 105. Air may be drawn into air inlet 105 by compression system 200. Compression system 200 may draw in air from the surroundings of the oxygen concentrator and compress the air, forcing the compressed air into one or both canisters 302 and 304. In an implementation, an inlet muffler 108 may be coupled to air inlet 105 to reduce sound produced by air being pulled into the oxygen concentrator by compression system 200. In an implementation, inlet muffler 108 may reduce moisture and sound. For example, a water adsorbent material (such as a polymer water adsorbent material or a zeolite material) may be used to both adsorb water from the incoming air and to reduce the sound of the air passing into the air inlet 105.

Compression system 200 may include one or more compressors configured to compress air. Pressurized air, produced by compression system 200, may be forced into one or both of the canisters 302 and 304. In some implementations, the ambient air may be pressurized in the canisters to a pressure approximately in a range of 13-20 pounds per square inch gauge pressure (psig). Other pressures may also be used, depending on the type of gas separation adsorbent disposed in the canisters.

Coupled to each canister 302/304 are inlet valves 122/124 and outlet valves 132/134. As shown in FIG. 1B, inlet valve 122 is coupled to canister 302 and inlet valve 124 is coupled to canister 304. Outlet valve 132 is coupled to canister 302 and outlet valve 134 is coupled to canister 304. Inlet valves 122/124 are used to control the passage of air from compression system 200 to the respective canisters. Outlet valves 132/134 are used to release gas from the respective canisters during a venting process. In some implementations, inlet valves 122/124 and outlet valves 132/134 may be silicon plunger solenoid valves. Other types of valves, however, may be used. Plunger valves offer advantages over other kinds of valves by being quiet and having low slippage.

In some implementations, a two-step valve actuation voltage may be generated to control inlet valves 122/124 and outlet valves 132/134. For example, a high voltage (e.g., 24 V) may be applied to an inlet valve to open the inlet valve. The voltage may then be reduced (e.g., to 7 V) to keep the inlet valve open. Using less voltage to keep a valve open may use less power (Power=Voltage*Current). This reduction in voltage minimizes heat buildup and power consumption to extend run time from the power supply 180 (described below). When the power is cut off to the valve, it closes by spring action. In some implementations, the voltage may be applied as a function of time that is not necessarily a stepped response (e.g., a curved downward voltage between an initial 24 V and a final 7 V).

In an implementation, pressurized air is sent into one of canisters 302 or 304 while the other canister is being vented. For example, during use, inlet valve 122 is opened while inlet valve 124 is closed. Pressurized air from compression system 200 is forced into canister 302, while being inhibited from entering canister 304 by inlet valve 124. In an implementation, a controller 400 is electrically coupled to valves 122, 124, 132, and 134. Controller 400 includes one or more processors 410 operable to execute program instructions stored in memory 420. The program instructions configure the controller to perform various predefined methods that are used to operate the oxygen concentrator, such as the methods described in more detail herein. The program instructions may include program instructions for operating inlet valves 122 and 124 out of phase with each other (i.e., when one of inlet valves 122 or 124 is opened, the other valve is closed). During pressurization of canister 302, outlet valve 132 is closed and outlet valve 134 is opened. Similar to the inlet valves, outlet valves 132 and 134 are operated out of phase with each other. In some implementations, the voltages and the durations of the voltages used to open the input and output valves may be controlled by controller 400. The controller 400 may include a transceiver 430 that may communicate with external devices to transmit data collected by the processor 410 or receive instructions from an external device for the processor 410.

Check valves 142 and 144 are coupled to canisters 302 and 304, respectively. Check valves 142 and 144 may be one-way valves that are passively operated by the pressure differentials that occur as the canisters are pressurized and vented, or may be active valves. Check valves 142 and 144 are coupled to the canisters to allow oxygen enriched air produced during pressurization of each canister to flow out of the canister, and to inhibit back flow of oxygen enriched air or any other gases into the canister. In this manner, check valves 142 and 144 act as one-way valves allowing oxygen enriched air to exit the respective canisters during pressurization.

The term “check valve”, as used herein, refers to a valve that allows flow of a fluid (gas or liquid) in one direction and inhibits back flow of the fluid. Examples of check valves that are suitable for use include, but are not limited to: a ball check valve; a diaphragm check valve; a butterfly check valve; a swing check valve; a duckbill valve; an umbrella valve; and a lift check valve. Under pressure, nitrogen molecules in the pressurized ambient air are adsorbed by the gas separation adsorbent in the pressurized canister. As the pressure increases, more nitrogen is adsorbed until the gas in the canister is enriched in oxygen. The non-adsorbed gas molecules (mainly oxygen) flow out of the pressurized canister when the pressure reaches a point sufficient to overcome the resistance of the check valve coupled to the canister. In one implementation, the pressure drop of the check valve in the forward direction is less than 1 psi. The break pressure in the reverse direction is greater than 100 psi. It should be understood, however, that modification of one or more components would alter the operating parameters of these valves. If the forward flow pressure is increased, there is, generally, a reduction in oxygen enriched air production. If the break pressure for reverse flow is reduced or set too low, there is, generally, a reduction in oxygen enriched air pressure.

In an exemplary implementation, canister 302 is pressurized by compressed air produced in compression system 200 and passed into canister 302. During pressurization of canister 302 inlet valve 122 is open, outlet valve 132 is closed, inlet valve 124 is closed and outlet valve 134 is open. Outlet valve 134 is opened when outlet valve 132 is closed to allow substantially simultaneous venting of canister 304 to atmosphere while canister 302 is being pressurized. Canister 302 is pressurized until the pressure in canister is sufficient to open check valve 142. Oxygen enriched air produced in canister 302 exits through check valve and, in one implementation, is collected in an accumulator 106.

After some time, the gas separation adsorbent will become saturated with nitrogen and will be unable to separate significant amounts of nitrogen from incoming air. This point is usually reached after a predetermined time of oxygen enriched air production. In the implementation described above, when the gas separation adsorbent in canister 302 reaches this saturation point, the inflow of compressed air is stopped and canister 302 is vented to remove nitrogen. During venting, inlet valve 122 is closed, and outlet valve 132 is opened. While canister 302 is being vented, canister 304 is pressurized to produce oxygen enriched air in the same manner described above. Pressurization of canister 304 is achieved by closing outlet valve 134 and opening inlet valve 124. The oxygen enriched air exits canister 304 through check valve 144.

During venting of canister 302, outlet valve 132 is opened allowing pressurized gas (mainly nitrogen) to exit the canister to atmosphere through concentrator outlet 130. In an implementation, the vented gases may be directed through muffler 133 to reduce the noise produced by releasing the pressurized gas from the canister. As gas is released from canister 302, the pressure in the canister 302 drops, allowing the nitrogen to become desorbed from the gas separation adsorbent. The released nitrogen exits the canister through outlet 130, resetting the canister to a state that allows renewed separation of nitrogen from an air stream. Muffler 133 may include open cell foam (or another material) to muffle the sound of the gas leaving the oxygen concentrator. In some implementations, the combined muffling components/techniques for the input of air and the output of oxygen enriched air may provide for oxygen concentrator operation at a sound level below 50 decibels.

During venting of the canisters, it is advantageous that at least a majority of the nitrogen is removed. In an implementation, at least about 50%, at least about 60%, at least about 70%, at least about 80%, at least about 90%, at least about 95%, at least about 98%, or substantially all of the nitrogen in a canister is removed before the canister is re-used to separate nitrogen from air. In some implementations, nitrogen removal may be assisted using an oxygen enriched air stream that is introduced into the canister from the other canister or stored oxygen enriched air.

In an exemplary implementation, a portion of the oxygen enriched air may be transferred from canister 302 to canister 304 when canister 304 is being vented of nitrogen. Transfer of oxygen enriched air from canister 302 to canister 304 during venting of canister 304 helps to desorb nitrogen from the adsorbent by lowering the partial pressure of nitrogen adjacent the adsorbent. The flow of oxygen enriched air also helps to purge desorbed nitrogen (and other gases) from the canister. In an implementation, oxygen enriched air may travel through flow restrictors 151, 153, and 155 between the two canisters. Flow restrictor 151 may be a trickle flow restrictor. Flow restrictor 151, for example, may be a 0.009 D flow restrictor (e.g., the flow restrictor has a radius 0.009″ which is less than the diameter of the tube it is inside). Flow restrictors 153 and 155 may be 0.013 D flow restrictors. Other flow restrictor types and sizes are also contemplated and may be used depending on the specific configuration and tubing used to couple the canisters. In some implementations, the flow restrictors may be press fit flow restrictors that restrict air flow by introducing a narrower diameter in their respective tube. In some implementations, the press fit flow restrictors may be made of sapphire, metal or plastic (other materials are also contemplated).

Flow of oxygen enriched air between the canisters is also controlled by use of valve 152 and valve 154. Valves 152 and 154 may be opened for a short duration during the venting process (and may be closed otherwise) to prevent excessive oxygen loss out of the purging canister. Other durations are also contemplated. In an exemplary implementation, canister 302 is being vented and it is desirable to purge canister 302 by passing a portion of the oxygen enriched air being produced in canister 304 into canister 302. A portion of oxygen enriched air, upon pressurization of canister 304, will pass through flow restrictor 151 into canister 302 during venting of canister 302. Additional oxygen enriched air is passed into canister 302, from canister 304, through valve 154 and flow restrictor 155. Valve 152 may remain closed during the transfer process, or may be opened if additional oxygen enriched air is needed. The selection of appropriate flow restrictors 151 and 155, coupled with controlled opening of valve 154 allows a controlled amount of oxygen enriched air to be sent from canister 304 to canister 302. In an implementation, the controlled amount of oxygen enriched air is an amount sufficient to purge canister 302 and minimize the loss of oxygen enriched air through venting valve 132 of canister 302. While this implementation describes venting of canister 302, it should be understood that the same process can be used to vent canister 304 using flow restrictor 151, valve 152 and flow restrictor 153.

The pair of equalization/vent valves 152/154 work with flow restrictors 153 and 155 to optimize the gas flow balance between the two canisters. This may allow for better flow control for venting one of the canisters with oxygen enriched air from the other of the canisters. It may also provide better flow direction between the two canisters. It has been found that, while flow valves 152/154 may be operated as bi-directional valves, the flow rate through such valves varies depending on the direction of fluid flowing through the valve. For example, oxygen enriched air flowing from canister 304 toward canister 302 has a flow rate faster through valve 152 than the flow rate of oxygen enriched air flowing from canister 302 toward canister 304 through valve 152. If a single valve was to be used, eventually either too much or too little oxygen enriched air would be sent between the canisters and the canisters would, over time, begin to produce different amounts of oxygen enriched air. Use of opposing valves and flow restrictors on parallel air pathways may equalize the flow pattern of the oxygen enriched air between the two canisters. Equalizing the flow may allow for a steady amount of oxygen enriched air to be available to the user over multiple cycles and also may allow a predictable volume of oxygen enriched air to purge the other of the canisters. In some implementations, the air pathway may not have restrictors but may instead have a valve with a built-in resistance or the air pathway itself may have a narrow radius to provide resistance.

At times, oxygen concentrator may be shut down for a period of time. When an oxygen concentrator is shut down, the temperature inside the canisters may drop as a result of the loss of adiabatic heat from the compression system. As the temperature drops, the volume occupied by the gases inside the canisters will drop. Cooling of the canisters may lead to a negative pressure in the canisters. Valves (e.g., valves 122, 124, 132, and 134) leading to and from the canisters are dynamically sealed rather than hermetically sealed. Thus, outside air may enter the canisters after shutdown to accommodate the pressure differential. When outside air enters the canisters, moisture from the outside air may be adsorbed by the gas separation adsorbent. Adsorption of water inside the canisters may lead to gradual degradation of the gas separation adsorbents, steadily reducing ability of the gas separation adsorbents to produce oxygen enriched air.

In an implementation, outside air may be inhibited from entering canisters after the oxygen concentrator is shut down by pressurizing both canisters prior to shutdown. By storing the canisters under a positive pressure, the valves may be forced into a hermetically closed position by the internal pressure of the air in the canisters. In an implementation, the pressure in the canisters, at shutdown, should be at least greater than ambient pressure. As used herein the term “ambient pressure” refers to the pressure of the surroundings in which the oxygen concentrator is located (e.g., the pressure inside a room, outside, in a plane, etc.). In an implementation, the pressure in the canisters, at shutdown, is at least greater than standard atmospheric pressure (i.e., greater than 760 mmHg (Ton), 1 atm, 101,325 Pa). In an implementation, the pressure in the canisters, at shutdown, is at least about 1.1 times greater than ambient pressure; is at least about 1.5 times greater than ambient pressure; or is at least about 2 times greater than ambient pressure.

In an implementation, pressurization of the canisters may be achieved by directing pressurized air into each canister from the compression system and closing all valves to trap the pressurized air in the canisters. In an exemplary implementation, when a shutdown sequence is initiated, inlet valves 122 and 124 are opened and outlet valves 132 and 134 are closed. Because inlet valves 122 and 124 are joined together by a common conduit, both canisters 302 and 304 may become pressurized as air and/or oxygen enriched air from one canister may be transferred to the other canister. This situation may occur when the pathway between the compression system and the two inlet valves allows such transfer. Because the oxygen concentrator operates in an alternating pressurize/venting mode, at least one of the canisters should be in a pressurized state at any given time. In an alternate implementation, the pressure may be increased in each canister by operation of compression system 200. When inlet valves 122 and 124 are opened, pressure between canisters 302 and 304 will equalize, however, the equalized pressure in either canister may not be sufficient to inhibit air from entering the canisters during shutdown. In order to ensure that air is inhibited from entering the canisters, compression system 200 may be operated for a time sufficient to increase the pressure inside both canisters to a level at least greater than ambient pressure. Regardless of the method of pressurization of the canisters, once the canisters are pressurized, inlet valves 122 and 124 are closed, trapping the pressurized air inside the canisters, which inhibits air from entering the canisters during the shutdown period.

Referring to FIG. 1C, an implementation of an oxygen concentrator 100 is depicted. Oxygen concentrator 100 includes a compression system 200, a canister system 300, and a power supply 180 disposed within an outer housing 170. Inlets 101 are located in outer housing 170 to allow air from the environment to enter oxygen concentrator 100. Inlets 101 may allow air to flow into the compartment to assist with cooling of the components in the compartment. Power supply 180 provides a source of power for the oxygen concentrator 100. Compression system 200 draws air in through the inlet 105 and muffler 108. Muffler 108 may reduce noise of air being drawn in by the compression system and also may include a desiccant material to remove water from the incoming air. Oxygen concentrator 100 may further include fan 172 used to vent air and other gases from the oxygen concentrator via outlet 173.

3. Compression System

In some implementations, compression system 200 includes one or more compressors. In another implementation, compression system 200 includes a single compressor, coupled to all of the canisters of canister system 300. Turning to FIGS. 1D and 1E, a compression system 200 is depicted that includes compressor 210 and motor 220. Motor 220 is coupled to compressor 210 and provides an operating force to the compressor to operate the compression mechanism. For example, motor 220 may be a motor providing a rotating component that causes cyclical motion of a component of the compressor that compresses air. When compressor 210 is a piston type compressor, motor 220 provides an operating force which causes the piston of compressor 210 to be reciprocated. Reciprocation of the piston causes compressed air to be produced by compressor 210. The pressure of the compressed air is, in part, estimated by the speed that the compressor is operated at, (e.g., how fast the piston is reciprocated). Motor 220, therefore, may be a variable speed motor that is operable at various speeds to dynamically control the pressure of air produced by compressor 210.

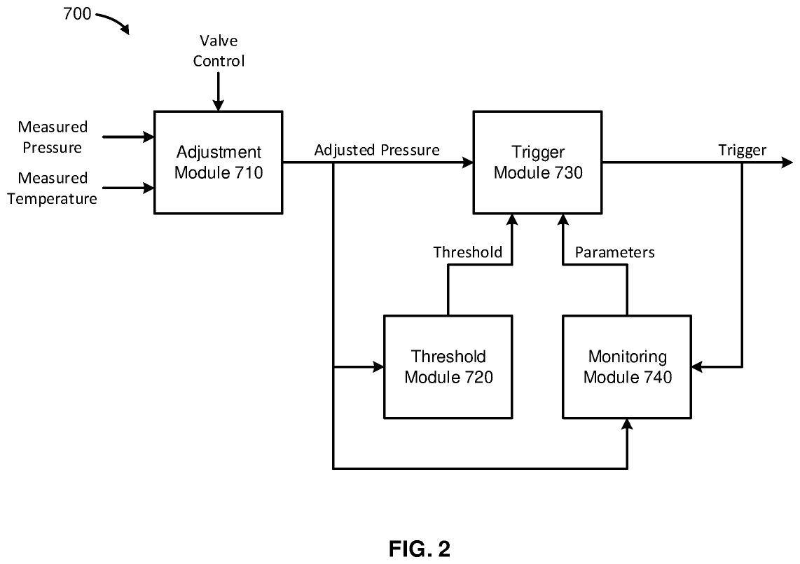

In one implementation, compressor 210 includes a single head wobble type compressor having a piston. Other types of compressors may be used such as diaphragm compressors and other types of piston compressors. Motor 220 may be a DC or AC motor and provides the operating power to the compressing component of compressor 210. Motor 220, in an implementation, may be a brushless DC motor. Motor 220 may be a variable speed motor configured to operate the compressing component of compressor 210 at variable speeds. Motor 220 may be coupled to controller 400, as depicted in FIG. 1B, which sends operating signals to the motor to control the operation of the motor. For example, controller 400 may send signals to motor 220 to: turn the motor on, turn motor the off, and set the operating speed of motor. Thus, as illustrated in FIG. 2, the compression system may include a speed sensor 201. The speed sensor may be a motor speed transducer used to determine a rotational velocity of the motor 220 and/or other reciprocating operation of the compression system 200. For example, a motor speed signal from the motor speed transducer may be provided to the controller 400. The speed sensor or motor speed transducer may, for example, be a Hall effect sensor. The controller 400 may operate the compression system via the motor 220 based on the speed signal and/or any other sensor signal of the oxygen concentrator, such as a pressure sensor (e.g., accumulator pressure sensor 107). Thus, as illustrated in FIG. 2, the controller 400 receives sensor signals, such as a speed signal from the speed sensor 201 and accumulator pressure signal from the accumulator pressure sensor 107. With such signal(s), the controller may implement one or more control loops (e.g., feedback control) for operation of the compression system based on sensor signals such as accumulator pressure and/or motor speed as described in more detail herein.

Compression system 200 inherently creates substantial heat. Heat is caused by the consumption of power by motor 220 and the conversion of power into mechanical motion. Compressor 210 generates heat due to the increased resistance to movement of the compressor components by the air being compressed. Heat is also inherently generated due to adiabatic compression of the air by compressor 210. Thus, the continual pressurization of air produces heat in the enclosure. Additionally, power supply 180 may produce heat as power is supplied to compression system 200. Furthermore, users of the oxygen concentrator may operate the device in unconditioned environments (e.g., outdoors) at potentially higher ambient temperatures than indoors, thus the incoming air will already be in a heated state.

Heat produced inside oxygen concentrator 100 can be problematic. Lithium ion batteries are generally employed as power supplies for oxygen concentrators due to their long life and light weight. Lithium ion battery packs, however, are dangerous at elevated temperatures and safety controls are employed in oxygen concentrator 100 to shutdown the system if dangerously high power supply temperatures are detected. Additionally, as the internal temperature of oxygen concentrator 100 increases, the amount of oxygen generated by the concentrator may decrease. This is due, in part, to the decreasing amount of oxygen in a given volume of air at higher temperatures. If the amount of produced oxygen drops below a predetermined amount, the oxygen concentrator 100 may automatically shut down.

Because of the compact nature of oxygen concentrators, dissipation of heat can be difficult. Solutions typically involve the use of one or more fans to create a flow of cooling air through the enclosure. Such solutions, however, require additional power from the power supply 180 and thus shorten the portable usage time of the oxygen concentrator. In an implementation, a passive cooling system may be used that takes advantage of the mechanical power produced by motor 220. Referring to FIGS. 1D and 1E, compression system 200 includes motor 220 having an external rotating armature 230. Specifically, armature 230 of motor 220 (e.g., a DC motor) is wrapped around the stationary field that is driving the armature. Since motor 220 is a large contributor of heat to the overall system it is helpful to transfer heat off the motor and sweep it out of the enclosure. With the external high speed rotation, the relative velocity of the major component of the motor and the air in which it exists is very high. The surface area of the armature is larger if externally mounted than if it is internally mounted. Since the rate of heat exchange is proportional to the surface area and the square of the velocity, using a larger surface area armature mounted externally increases the ability of heat to be dissipated from motor 220. The gain in cooling efficiency by mounting the armature externally, allows the elimination of one or more cooling fans, thus reducing the weight and power consumption while maintaining the interior of the oxygen concentrator within the appropriate temperature range. Additionally, the rotation of the externally mounted armature creates movement of air proximate to the motor to create additional cooling.

Moreover, an external rotating armature may help the efficiency of the motor, allowing less heat to be generated. A motor having an external armature operates similar to the way a flywheel works in an internal combustion engine. When the motor is driving the compressor, the resistance to rotation is low at low pressures. When the pressure of the compressed air is higher, the resistance to rotation of the motor is higher. As a result, the motor does not maintain consistent ideal rotational stability, but instead surges and slows down depending on the pressure demands of the compressor. This tendency of the motor to surge and then slow down is inefficient and therefore generates heat. Use of an external armature adds greater angular momentum to the motor which helps to compensate for the variable resistance experienced by the motor. Since the motor does not have to work as hard, the heat produced by the motor may be reduced.

In an implementation, cooling efficiency may be further increased by coupling an air transfer device 240 to external rotating armature 230. In an implementation, air transfer device 240 is coupled to the external armature 230 such that rotation of the external armature 230 causes the air transfer device 240 to create an air flow that passes over at least a portion of the motor. In an implementation, air transfer device 240 includes one or more fan blades coupled to the external armature 230. In an implementation, a plurality of fan blades may be arranged in an annular ring such that the air transfer device 240 acts as an impeller that is rotated by movement of the external rotating armature 230. As depicted in FIGS. 1D and 1E, air transfer device 240 may be mounted to an outer surface of the external armature 230, in alignment with the motor 220. The mounting of the air transfer device 240 to the armature 230 allows air flow to be directed toward the main portion of the external rotating armature 230, providing a cooling effect during use. In an implementation, the air transfer device 240 directs air flow such that a majority of the external rotating armature 230 is in the air flow path.

Further, referring to FIGS. 1D and 1E, air pressurized by compressor 210 exits compressor 210 at compressor outlet 212. A compressor outlet conduit 250 is coupled to compressor outlet 212 to transfer the compressed air to canister system 300. As noted previously, compression of air causes an increase in the temperature of the air. This increase in temperature can be detrimental to the efficiency of the oxygen concentrator. In order to reduce the temperature of the pressurized air, compressor outlet conduit 250 is placed in the air flow path produced by air transfer device 240. At least a portion of compressor outlet conduit 250 may be positioned proximate to motor 220. Thus, air flow, created by air transfer device 240 may contact both motor 220 and compressor outlet conduit 250. In one implementation, a majority of compressor outlet conduit 250 is positioned proximate to motor 220. In an implementation, the compressor outlet conduit 250 is coiled around motor 220, as depicted in FIG. 1E.

In an implementation, the compressor outlet conduit 250 is composed of a heat exchange metal. Heat exchange metals include, but are not limited to, aluminum, carbon steel, stainless steel, titanium, copper, copper-nickel alloys or other alloys formed from combinations of these metals. Thus, compressor outlet conduit 250 can act as a heat exchanger to remove heat that is inherently caused by compression of the air. By removing heat from the compressed air, the number of molecules in a given volume at a given pressure is increased. As a result, the amount of oxygen enriched air that can be generated by each canister during each pressure swing cycle may be increased.

The heat dissipation mechanisms described herein are either passive or make use of elements required for the oxygen concentrator 100. Thus, for example, dissipation of heat may be increased without using systems that require additional power. By not requiring additional power, the run-time of the battery packs may be increased and the size and weight of the oxygen concentrator may be minimized. Likewise, use of an additional box fan or cooling unit may be eliminated. Eliminating such additional features reduces the weight and power consumption of the oxygen concentrator.

As discussed above, adiabatic compression of air causes the air temperature to increase. During venting of a canister in canister system 300, the pressure of the gas being released from the canisters decreases. The adiabatic decompression of the gas in the canister causes the temperature of the gas to drop as it is vented. In an implementation, the cooled vented gases 327 from canister system 300 are directed toward power supply 180 and toward compression system 200. In an implementation, base 315 of canister system 300 receives the vented gases from the canisters. The vented gases 327 are directed through base 315 toward outlet 325 of the base and toward power supply 180. The vented gases, as noted, are cooled due to decompression of the gases and therefore passively provide cooling to the power supply 180. When the compression system is operated, the air transfer device 240 will gather the cooled vented gases and direct the gases toward the motor of compression system 200. Fan 172 may also assist in directing the vented gas across compression system 200 and out of the housing 170. In this manner, additional cooling may be obtained without requiring any further power from the battery.

4. Canister System

Oxygen concentrator system 100 may include at least two canisters, each canister including a gas separation adsorbent. The canisters of oxygen concentrator system 100 may be disposed formed from a molded housing. In an implementation, canister system 300 includes two housing components 310 and 510, as depicted in FIG. 1I. In various implementations, the housing components 310 and 510 of the oxygen concentrator 100 may form a two-part molded plastic frame that defines two canisters 302 and 304 and accumulator 106. The housing components 310 and 510 may be formed separately and then coupled together. In some implementations, housing components 310 and 510 may be injection molded or compression molded. Housing components 310 and 510 may be made from a thermoplastic polymer such as polycarbonate, methylene carbide, polystyrene, acrylonitrile butadiene styrene (ABS), polypropylene, polyethylene, or polyvinyl chloride. In another implementation, housing components 310 and 510 may be made of a thermoset plastic or metal (such as stainless steel or a lightweight aluminum alloy). Lightweight materials may be used to reduce the weight of the oxygen concentrator 100. In some implementations, the two housings 310 and 510 may be fastened together using screws or bolts. Alternatively, housing components 310 and 510 may be solvent welded together.

As shown, valve seats 322, 324, 332, and 334 and air pathways 330 and 346 may be integrated into the housing component 310 to reduce the number of sealed connections needed throughout the air flow of the oxygen concentrator 100.

Air pathways/tubing between different sections in housing components 310 and 510 may take the form of molded conduits. Conduits in the form of molded channels for air pathways may occupy multiple planes in housing components 310 and 510. For example, the molded air conduits may be formed at different depths and at different x,y,z positions in housing components 310 and 510. In some implementations, a majority or substantially all of the conduits may be integrated into the housing components 310 and 510 to reduce potential leak points.

In some implementations, prior to coupling housing components 310 and 510 together, O-rings may be placed between various points of housing components 310 and 510 to ensure that the housing components are properly sealed. In some implementations, components may be integrated and/or coupled separately to housing components 310 and 510. For example, tubing, flow restrictors (e.g., press fit flow restrictors), oxygen sensors, gas separation adsorbents, check valves, plugs, processors, power supplies, etc. may be coupled to housing components 310 and 510 before and/or after the housing components are coupled together.

In some implementations, apertures 337 leading to the exterior of housing components 310 and 510 may be used to insert devices such as flow restrictors. Apertures may also be used for increased moldability. One or more of the apertures may be plugged after molding (e.g., with a plastic plug). In some implementations, flow restrictors may be inserted into passages prior to inserting plugs to seal the passages. Press fit flow restrictors may have diameters that may allow a friction fit between the press fit flow restrictors and their respective apertures. In some implementations, an adhesive may be added to the exterior of the press fit flow restrictors to hold the press fit flow restrictors in place once inserted. In some implementations, the plugs may have a friction fit with their respective tubes (or may have an adhesive applied to their outer surface). The press fit flow restrictors and/or other components may be inserted and pressed into their respective apertures using a narrow tip tool or rod (e.g., with a diameter less than the diameter of the respective aperture). In some implementations, the press fit flow restrictors may be inserted into their respective tubes until they abut a feature in the tube to halt their insertion. For example, the feature may include a reduction in radius. Other features are also contemplated (e.g., a bump in the side of the tubing, threads, etc.). In some implementations, press fit flow restrictors may be molded into the housing components (e.g., as narrow tube segments).

In some implementations, spring baffle 139 may be placed into respective canister receiving portions of housing components 310 and 510 with the spring side of the baffle 139 facing the exit of the canister. Spring baffle 139 may apply force to gas separation adsorbent in the canister while also assisting in preventing gas separation adsorbent from entering the exit apertures. Use of a spring baffle 139 may keep the gas separation adsorbent compact while also allowing for expansion (e.g., thermal expansion). Keeping the gas separation adsorbent compact may prevent the gas separation adsorbent from breaking during movement of the oxygen concentrator system 100.

In some implementations, filter 129 may be placed into respective canister receiving portions of housing components 310 and 510 facing the inlet of the respective canisters. The filter 129 removes particles from the feed gas stream entering the canisters.

In some implementations, pressurized air from the compression system 200 may enter air inlet 306. Air inlet 306 is coupled to inlet conduit 330. Air enters housing component 310 through inlet 306 and travels through conduit 330, and then to valve seats 322 and 324. FIG. 1J and FIG. 1K depict an end view of housing 310. FIG. 1J depicts an end view of housing 310 prior to fitting valves to housing 310. FIG. 1K depicts an end view of housing 310 with the valves fitted to the housing 310. Valve seats 322 and 324 are configured to receive inlet valves 122 and 124 respectively. Inlet valve 122 is coupled to canister 302 and inlet valve 124 is coupled to canister 304. Housing 310 also includes valve seats 332 and 334 configured to receive outlet valves 132 and 134 respectively. Outlet valve 132 is coupled to canister 302 and outlet valve 134 is coupled to canister 304. Inlet valves 122/124 are used to control the passage of air from conduit 330 to the respective canisters.

In an implementation, pressurized air is sent into one of canisters 302 or 304 while the other canister is being vented. For example, during use, inlet valve 122 is opened while inlet valve 124 is closed. Pressurized air from compression system 200 is forced into canister 302, while being inhibited from entering canister 304 by inlet valve 124. During pressurization of canister 302, outlet valve 132 is closed and outlet valve 134 is opened. Similar to the inlet valves, outlet valves 132 and 134 are operated out of phase with each other. Valve seat 322 includes an opening 323 that passes through housing 310 into canister 302. Similarly valve seat 324 includes an opening 375 that passes through housing 310 into canister 302. Air from conduit 330 passes through openings 323 or 375 if the respective valves 322 and 324 are open, and enters a canister.

Check valves 142 and 144 (See FIG. 1I) are coupled to canisters 302 and 304, respectively. Check valves 142 and 144 are one-way valves that are passively operated by the pressure differentials that occur as the canisters are pressurized and vented. Oxygen enriched air produced in canisters 302 and 304 passes from the canisters into openings 542 and 544 of housing component 510. A passage (not shown) links openings 542 and 544 to conduits 342 and 344, respectively. Oxygen enriched air produced in canister 302 passes from the canister though opening 542 and into conduit 342 when the pressure in the canister is sufficient to open check valve 142. When check valve 142 is open, oxygen enriched air flows through conduit 342 toward the end of housing 310. Similarly, oxygen enriched air produced in canister 304 passes from the canister through opening 544 and into conduit 344 when the pressure in the canister is sufficient to open check valve 144. When check valve 144 is open, oxygen enriched air flows through conduit 344 toward the end of housing 310.

Oxygen enriched air from either canister travels through conduit 342 or 344 and enters conduit 346 formed in housing 310. Conduit 346 includes openings that couple the conduit to conduit 342, conduit 344 and accumulator 106. Thus, oxygen enriched air, produced in canister 302 or 304, travels to conduit 346 and passes into accumulator 106. As illustrated in FIG. 1B, gas pressure within the accumulator 106 may be measured by a sensor, such as with an accumulator pressure sensor 107. (See also FIG. 1F.) Thus, the accumulator pressure sensor provides a signal representing the pressure of the accumulated oxygen enriched air. An example of a suitable pressure transducer is a sensor from the HONEYWELL ASDX series. An alternative suitable pressure transducer is a sensor from the NPA Series from GENERAL ELECTRIC. In some versions, the pressure sensor may alternatively measure pressure of the gas outside of the accumulator 106, such as in an output path between the accumulator 106 and a valve (e.g., supply valve 160) that controls the release of the oxygen enriched air for delivery to a user in a bolus.

After some time, the gas separation adsorbent will become saturated with nitrogen and will be unable to separate significant amounts of nitrogen from incoming air. When the gas separation adsorbent in a canister reaches this saturation point, the inflow of compressed air is stopped and the canister is vented to remove nitrogen. Canister 302 is vented by closing inlet valve 122 and opening outlet valve 132. Outlet valve 132 releases the vented gas from canister 302 into the volume defined by the end of housing 310. Foam material may cover the end of housing 310 to reduce the sound made by release of gases from the canisters. Similarly, canister 304 is vented by closing inlet valve 124 and opening outlet valve 134. Outlet valve 134 releases the vented gas from canister 304 into the volume defined by the end of housing 310.

While canister 302 is being vented, canister 304 is pressurized to produce oxygen enriched air in the same manner described above. Pressurization of canister 304 is achieved by closing outlet valve 134 and opening inlet valve 124. The oxygen enriched air exits canister 304 through check valve 144.

In an exemplary implementation, a portion of the oxygen enriched air may be transferred from canister 302 to canister 304 when canister 304 is being vented of nitrogen. Transfer of oxygen enriched air from canister 302 to canister 304, during venting of canister 304, helps to desorb nitrogen from the adsorbent by lowering the partial pressure of nitrogen adjacent the adsorbent. The flow of oxygen enriched air also helps to purge desorbed nitrogen (and other gases) from the canister. Flow of oxygen enriched air between the canisters is controlled using flow restrictors and valves, as depicted in FIG. 1B. Three conduits are formed in housing component 510 for use in transferring oxygen enriched air between canisters. As shown in FIG. 1L, conduit 530 couples canister 302 to canister 304. Flow restrictor 151 (not shown) is disposed in conduit 530, between canister 302 and canister 304 to restrict flow of oxygen enriched air during use. Conduit 532 also couples canister 302 to 304. Conduit 532 is coupled to valve seat 552 which receives valve 152, as shown in FIG. 1M. Flow restrictor 153 (not shown) is disposed in conduit 532, between canister 302 and 304. Conduit 534 also couples canister 302 to 304. Conduit 534 is coupled to valve seat 554 which receives valve 154, as shown in FIG. 1M. Flow restrictor 155 (not shown) is disposed in conduit 534, between canister 302 and 304. The pair of equalization/vent valves 152/154 work with flow restrictors 153 and 155 to optimize the air flow balance between the two canisters.

Oxygen enriched air in accumulator 106 passes through supply valve 160 into expansion chamber 162 which is formed in housing component 510. An opening (not shown) in housing component 510 couples accumulator 106 to supply valve 160. In an implementation, expansion chamber 162 may include one or more devices configured to estimate an oxygen concentration of gas passing through the chamber.

5. Outlet System

An outlet system, coupled to one or more of the canisters, includes one or more conduits for providing oxygen enriched air to a user. In an implementation, oxygen enriched air produced in either of canisters 302 and 304 is collected in accumulator 106 through check valves 142 and 144, respectively, as depicted schematically in FIG. 1B. The oxygen enriched air leaving the canisters may be collected in accumulator 106 prior to being provided to a user. In some implementations, a tube may be coupled to accumulator 106 to provide the oxygen enriched air to the user. Oxygen enriched air may be provided to the user through an airway delivery device that transfers the oxygen enriched air to the user's mouth and/or nose. In an implementation, an outlet may include a tube that directs the oxygen toward a user's nose and/or mouth that may not be directly coupled to the user's nose.

Turning to FIG. 1F, a schematic diagram of an implementation of an outlet system for an oxygen concentrator is shown. A supply valve 160 may be coupled to an outlet tube to control the release of the oxygen enriched air from accumulator 106 to the user. In an implementation, supply valve 160 is an electromagnetically actuated plunger valve. Supply valve 160 is actuated by controller 400 to control the delivery of oxygen enriched air to a user. Actuation of supply valve 160 is not timed or synchronized to the pressure swing adsorption process. Instead, actuation is synchronized to the user's breathing as described below. In some implementations, supply valve 160 may have continuously-valued actuation to establish a clinically effective amplitude profile for providing oxygen enriched air.

Oxygen enriched air in accumulator 106 passes through supply valve 160 into expansion chamber 162 as depicted in FIG. 1F. In an implementation, expansion chamber 162 may include one or more devices configured to estimate an oxygen concentration of gas passing through the expansion chamber 162. Oxygen enriched air in expansion chamber 162 builds briefly, through release of gas from accumulator 106 by supply valve 160, and then is bled through a small orifice flow restrictor 175 to a flow rate sensor 185 and then to particulate filter 187. Flow restrictor 175 may be a 0.025 D flow restrictor. Other flow restrictor types and sizes may be used. In some implementations, the diameter of the air pathway in the housing may be restricted to create restricted gas flow. Flow rate sensor 185 may be any sensor configured to generate a signal representing the rate of gas flowing through the conduit. Particulate filter 187 may be used to filter bacteria, dust, granule particles, etc., prior to delivery of the oxygen enriched air to the user. The oxygen enriched air passes through filter 187 to connector 190 which sends the oxygen enriched air to the user via delivery conduit 192 and to pressure sensor 194. The fluid dynamics of the outlet pathway, coupled with the programmed actuations of supply valve 160, may result in a bolus of oxygen being provided at the correct time and with an amplitude profile that assures rapid delivery into the user's lungs without excessive waste.

Expansion chamber 162 may include one or more oxygen sensors adapted to determine an oxygen concentration of gas passing through the chamber. In an implementation, the oxygen concentration of gas passing through expansion chamber 162 is estimated using an oxygen sensor 165. An oxygen sensor is a device configured to measure oxygen concentration in a gas. Examples of oxygen sensors include, but are not limited to, ultrasonic oxygen sensors, electrical oxygen sensors, chemical oxygen sensors, and optical oxygen sensors. In one implementation, oxygen sensor 165 is an ultrasonic oxygen sensor that includes an ultrasonic emitter 166 and an ultrasonic receiver 168. In some implementations, ultrasonic emitter 166 may include multiple ultrasonic emitters and ultrasonic receiver 168 may include multiple ultrasonic receivers. In implementations having multiple emitters/receivers, the multiple ultrasonic emitters and multiple ultrasonic receivers may be axially aligned (e.g., across the gas flow path which may be perpendicular to the axial alignment).

In use, an ultrasonic sound wave from emitter 166 may be directed through oxygen enriched air disposed in chamber 162 to receiver 168. The ultrasonic oxygen sensor 165 may be configured to detect the speed of sound through the oxygen enriched air to determine the composition of the oxygen enriched air. The speed of sound is different in nitrogen and oxygen, and in a mixture of the two gases, the speed of sound through the mixture may be an intermediate value proportional to the relative amounts of each gas in the mixture. In use, the sound at the receiver 168 is slightly out of phase with the sound sent from emitter 166. This phase shift is due to the relatively slow velocity of sound through a gas medium as compared with the relatively fast speed of the electronic pulse through wire. The phase shift, then, is proportional to the distance between the emitter and the receiver and inversely proportional to the speed of sound through the expansion chamber 162. The density of the gas in the chamber affects the speed of sound through the expansion chamber and the density is proportional to the ratio of oxygen to nitrogen in the expansion chamber. Therefore, the phase shift can be used to measure the concentration of oxygen in the expansion chamber. In this manner the relative concentration of the oxygen in accumulator 106 may be estimated as a function of one or more properties of a detected sound wave traveling through the accumulator 106.

In some implementations, multiple emitters 166 and receivers 168 may be used. The readings from the emitters 166 and receivers 168 may be averaged to reduce errors that may be inherent in turbulent flow systems. In some implementations, the presence of other gases may also be detected by measuring the transit time and comparing the measured transit time to predetermined transit times for other gases and/or mixtures of gases.

The sensitivity of the ultrasonic sensor system may be increased by increasing the distance between the emitter 166 and receiver 168, for example to allow several sound wave cycles to occur between emitter 166 and the receiver 168. In some implementations, if at least two sound cycles are present, the influence of structural changes of the transducer may be reduced by measuring the phase shift relative to a fixed reference at two points in time. If the earlier phase shift is subtracted from the later phase shift, the shift caused by thermal expansion of expansion chamber 162 may be reduced or cancelled. The shift caused by a change of the distance between the emitter 166 and receiver 168 may be approximately the same at the measuring intervals, whereas a change owing to a change in oxygen concentration may be cumulative. In some implementations, the shift measured at a later time may be multiplied by the number of intervening cycles and compared to the shift between two adjacent cycles. Further details regarding sensing of oxygen in the expansion chamber may be found, for example, in U.S. patent application Ser. No. 12/163,549, entitled “Oxygen Concentrator Apparatus and Method,” which published as U.S. Publication No. 2009/0065007 A1 on Mar. 12, 2009 and is incorporated herein by reference.

Flow rate sensor 185 may be used to determine the flow rate of gas flowing through the outlet system. Flow rate sensors that may be used include, but are not limited to: diaphragm/bellows flow meters; rotary flow meters (e.g., Hall effect flow meters); turbine flow meters; orifice flow meters; and ultrasonic flow meters. Flow rate sensor 185 may be coupled to controller 400. The rate of gas flowing through the outlet system may be an indication of the breathing volume of the user. Changes in the flow rate of gas flowing through the outlet system may also be used to determine a breathing rate of the user. Controller 400 may generate a control signal or trigger signal to control actuation of supply valve 160. Such control of actuation of the supply valve may be based on the breathing rate and/or breathing volume of the user, as estimated by flow rate sensor 185.

In some implementations, ultrasonic sensor 165 and, for example, flow rate sensor 185 may provide a measurement of an actual amount of oxygen being provided. For example, flow rate sensor 185 may measure a volume of gas (based on flow rate) provided and ultrasonic sensor 165 may provide the concentration of oxygen of the gas provided. These two measurements together may be used by controller 400 to determine an approximation of the actual amount of oxygen provided to the user.

Oxygen enriched air passes through flow rate sensor 185 to filter 187. Filter 187 removes bacteria, dust, granule particles, etc., prior to providing the oxygen enriched air to the user. The filtered oxygen enriched air passes through filter 187 to connector 190. Connector 190 may be a “Y” connector coupling the outlet of filter 187 to pressure sensor 194 and delivery conduit 192. Pressure sensor 194 may be used to monitor the pressure of the gas passing through delivery conduit 192 to the user. In some implementations, pressure sensor 194 is configured to generate a signal that is proportional to the amount of positive or negative pressure applied to a sensing surface. Changes in pressure, sensed by pressure sensor 194, may be used to determine a breathing rate of a user, as well as the onset of inhalation (also referred to as the trigger instant) as described below. Controller 400 may control actuation of supply valve 160 based on the breathing rate and/or onset of inhalation of the user. In an implementation, controller 400 may control actuation of supply valve 160 based on information provided by either or both of the flow rate sensor 185 and the pressure sensor 194.

Oxygen enriched air may be provided to a user through delivery conduit 192. In an implementation, delivery conduit 192 may be a silicone tube. Delivery conduit 192 may be coupled to a user using an airway delivery device, as depicted in FIGS. 1G and 1H. An airway delivery device may be any device capable of providing the oxygen enriched air to nasal cavities or oral cavities. Examples of airway delivery devices include, but are not limited to: nasal masks, nasal pillows, nasal prongs, nasal cannulas, and mouthpieces. A nasal cannula airway delivery device 196 is depicted in FIG. 1G. Nasal cannula airway delivery device 196 is positioned proximate to a user's airway (e.g., proximate to the user's mouth and or nose) to allow delivery of the oxygen enriched air to the user while allowing the user to breathe air from the surroundings.

In an alternate implementation, a mouthpiece may be used to provide oxygen enriched air to the user. As shown in FIG. 1H, a mouthpiece 198 may be coupled to oxygen concentrator 100. Mouthpiece 198 may be the only device used to provide oxygen enriched air to the user, or a mouthpiece may be used in combination with a nasal delivery device (e.g., a nasal cannula). As depicted in FIG. 1H, oxygen enriched air may be provided to a user through both nasal cannula airway delivery device 196 and mouthpiece 198.

Mouthpiece 198 is removably positionable in a user's mouth. In one implementation, mouthpiece 198 is removably couplable to one or more teeth in a user's mouth. During use, oxygen enriched air is directed into the user's mouth via the mouthpiece. Mouthpiece 198 may be a night guard mouthpiece which is molded to conform to the user's teeth. Alternatively, mouthpiece may be a mandibular repositioning device. In an implementation, at least a majority of the mouthpiece is positioned in a user's mouth during use.

During use, oxygen enriched air may be directed to mouthpiece 198 when a change in pressure is detected proximate to the mouthpiece. In one implementation, mouthpiece 198 may be coupled to a pressure sensor 194. When a user inhales air through the user's mouth, pressure sensor 194 may detect a drop in pressure proximate to the mouthpiece. Controller 400 of oxygen concentrator 100 may control release of a bolus of oxygen enriched air to the user at the onset of inhalation.

During typical breathing of an individual, inhalation may occur through the nose, through the mouth or through both the nose and the mouth. Furthermore, breathing may change from one passageway to another depending on a variety of factors. For example, during more active activities, a user may switch from breathing through their nose to breathing through their mouth, or breathing through their mouth and nose. A system that relies on a single mode of delivery (either nasal or oral), may not function properly if breathing through the monitored pathway is stopped. For example, if a nasal cannula is used to provide oxygen enriched air to the user, an inhalation sensor (e.g., a pressure sensor or flow rate sensor) is coupled to the nasal cannula to determine the onset of inhalation. If the user stops breathing through their nose, and switches to breathing through their mouth, the oxygen concentrator 100 may not know when to provide the oxygen enriched air since there is no feedback from the nasal cannula. Under such circumstances, oxygen concentrator 100 may increase the flow rate and/or increase the frequency of providing oxygen enriched air until the inhalation sensor detects an inhalation by the user. If the user switches between breathing modes often, the default mode of providing oxygen enriched air may cause the oxygen concentrator 100 to work harder, limiting the portable usage time of the system.

In an implementation, mouthpiece 198 is used in combination with nasal cannula airway delivery device 196 to provide oxygen enriched air to a user, as depicted in FIG. 1H. Both mouthpiece 198 and nasal cannula airway delivery device 196 are coupled to an inhalation sensor. In one implementation, mouthpiece 198 and nasal cannula airway delivery device 196 are coupled to the same inhalation sensor. In an alternate implementation, mouthpiece 198 and nasal cannula airway delivery device 196 are coupled to different inhalation sensors. In either implementation, the inhalation sensor(s) may detect the onset of inhalation from either the mouth or the nose. Oxygen concentrator 100 may be configured to provide oxygen enriched air to the delivery device (i.e. mouthpiece 198 or nasal cannula airway delivery device 196) proximate to which the onset of inhalation was detected. Alternatively, oxygen enriched air may be provided to both mouthpiece 198 and nasal cannula airway delivery device 196 if onset of inhalation is detected proximate either delivery device. The use of a dual delivery system, such as depicted in FIG. 1H may be particularly useful for users when they are sleeping and may switch between nose breathing and mouth breathing without conscious effort.

6. Controller System

Operation of oxygen concentrator 100 may be performed automatically using an internal controller 400 coupled to various components of the oxygen concentrator 100, as described herein. Controller 400 includes one or more processors 410 and internal memory 420, as depicted in FIG. 1B. Methods used to operate and monitor oxygen concentrator 100 may be implemented by program instructions stored in internal memory 420 or an external memory medium coupled to controller 400, and executed by one or more processors 410. A memory medium may include any of various types of memory devices or storage devices. The term “memory medium” is intended to include an installation medium (e.g., a Compact Disc Read Only Memory (CD-ROM), floppy disks, or tape device), a computer system memory or random access memory (e.g., Dynamic Random Access Memory (DRAM), Double Data Rate Random Access Memory (DDR RAM), Static Random Access Memory (SRAM), Extended Data Out Random Access Memory (EDO RAM), Random Access Memory (RAM), etc.), or a non-volatile memory such as a magnetic medium (e.g., a hard drive or optical storage). The memory medium may comprise other types of memory as well, or combinations thereof. In addition, the memory medium may be located proximate to the controller 400 by which the programs are executed, or may be located in an external computing device that connects to the controller 400 over a network, such as the Internet. In the latter instance, the external computing device may provide program instructions to the controller 400 for execution. The term “memory medium” may include two or more memory media that may reside in different locations (e.g., in different computing devices that are connected over a network).

In some implementations, controller 400 includes processor 410 that includes, for example, one or more field programmable gate arrays (FPGAs), microcontrollers, etc. included on a circuit board disposed in oxygen concentrator 100. Processor 410 is configured to execute programming instructions stored in memory 420. In some implementations, programming instructions may be built into processor 410 such that a memory external to the processor 410 may not be separately accessed (i.e., the memory 420 may be internal to the processor 410).

Processor 410 may be coupled to various components of oxygen concentrator 100, including, but not limited to compression system 200, one or more of the valves used to control fluid flow through the system (e.g., valves 122, 124, 132, 134, 152, 154, 160), oxygen sensor 165, pressure sensor 194, flow rate sensor 185, temperature sensors (not shown), fan 172, and any other component that may be electrically controlled. In some implementations, a separate processor (and/or memory) may be coupled to one or more of the components.

Controller 400 is configured (e.g., programmed by program instructions) to operate oxygen concentrator 100 and is further configured to monitor the oxygen concentrator 100, such as for malfunction states or other process information. For example, in one implementation, controller 400 is programmed to trigger an alarm if the system is operating and no breathing is detected by the user for a predetermined amount of time. For example, if controller 400 does not detect a breath for a period of 75 seconds, an alarm LED may be lit and/or an audible alarm may be sounded. If the user has truly stopped breathing, for example, during a sleep apnea episode, the alarm may be sufficient to awaken the user, causing the user to resume breathing. The action of breathing may be sufficient for controller 400 to reset this alarm function. Alternatively, if the system is accidentally left on when delivery conduit 192 is removed from the user, the alarm may serve as a reminder for the user to turn oxygen concentrator 100 off.

Controller 400 is further coupled to oxygen sensor 165, and may be programmed for continuous or periodic monitoring of the oxygen concentration of the oxygen enriched air passing through expansion chamber 162. A minimum oxygen concentration threshold may be programmed into controller 400, such that the controller lights an LED visual alarm and/or an audible alarm to warn the user of the low concentration of oxygen.

Controller 400 is also coupled to internal power supply 180 and may be configured to monitor the level of charge of the internal power supply. A minimum voltage and/or current threshold may be programmed into controller 400, such that the controller lights an LED visual alarm and/or an audible alarm to warn the user of low power condition. The alarms may be activated intermittently and at an increasing frequency as the battery approaches zero usable charge.

FIG. 1O illustrates one implementation of a connected respiratory therapy system 450 including the oxygen concentrator 100. Controller 400 of the oxygen concentrator 100 includes the transceiver 430 configured to allow the controller 400 to communicate, using a wireless communication protocol such as the Global System for Mobile Telephony (GSM) or other protocol (e.g., WiFi), with a remote computing device such as a cloud-based server 460 such as over a network 470. The network 470 may be a wide-area network such as the Internet, or a local-area network such as an Ethernet. The controller 400 may also include a short range wireless module in the transceiver 430 configured to enable the controller 400 to communicate, using a short range wireless communication protocol such as Bluetooth™, with a portable computing device 480 such as a smartphone. The portable computing device, e.g., smartphone, 480 may be associated with a user 1000 of the oxygen concentrator 100.

The server 460 may also be in wireless communication with the portable computing device 480 using a wireless communication protocol such as GSM. A processor of the smartphone 480 may execute a program 482 known as an “app” to control the interaction of the smartphone 480 with the user 1000, the oxygen concentrator 100, and/or the server 460. The server 460 may have access to a database 466 that stores operational data about the oxygen concentrator 100 and user 1000.

The server 460 includes an analysis engine 462 that may execute methods of operating and monitoring the oxygen concentrator 100 as further described below. The server 460 may also be in communication via the network 470 with other devices such as a personal computing device workstation 464 via a wired or wireless connection. A processor of the personal computing device 464 may execute a “client” program to control the interaction of the personal computing device 464 with the server 460. One example of a client program is a browser.

In a further implementation, the server 460 may be configured to host a portal system. The portal system may receive, from the portable computing device 480 or directly from the oxygen concentrator 100, data relating to the operation of the oxygen concentrator 100. As described above, the personal computing device 464 may execute a client program such as a browser to allow a user of the personal computing device 464 (such as a representative of an HME) to access the operational data of the oxygen concentrator 100, and other POCs in the connected respiratory therapy system 450, via the portal system hosted by the server 460. In this fashion, such a portal system may be utilized by an HME to manage a population of users of POC devices, e.g., the oxygen concentrator 100, in the connected respiratory therapy system 450. The portal system may provide actionable insights into user or device condition for the population of POC devices and their users based on the operational data received by the portal system. Such insights may be based on rules that are applied to the operational data.

Further functions that may be implemented with or by the controller 400 are described in detail in other sections of this disclosure. For example, the controller of the POC may implement compressor control to regulate pressure in the system. Thus, the POC may be equipped with a pressure sensor such as in the accumulator downstream of the sieve beds. The controller 400 in the POC can control adjusting of the speed of the compressor using signals from the pressure sensor as well as a motor speed sensor such as in one or more modes. In this regard, the controller may implement dual control modes, designated a coarse pressure regulation mode and a fine pressure regulation mode. The coarse pressure regulation mode may be implemented for changing between the different flow rate settings (or “flow settings”) of the POC and for starting/initial activation. The fine pressure regulation mode may then take over upon completion of each operation of the coarse pressure regulation mode.

In the coarse pressure regulation mode, the motor speed is set/controlled to ramp up or down depending the prior state of operations. During the ramping, the controller uses the measurements from the pressure sensor to generate an estimated pressure upstream of the sensor, in the sieve beds. In some implementations, the estimated pressure is used in a test to terminate the ramp, e.g., when the estimated pressure reaches a predetermined pressure target, created at manufacturing time, that is associated with the selected flow rate setting of the POC. The pressure estimate may be calculated by performing a regression (e.g., linear) using data from the pressure sensor whereby the controller determines regression parameters (e.g., slope and intercept parameters of a line) from the sensor signal samples. The pressure estimate is calculated with the regression parameters and a known system response delay.

In the fine pressure regulation mode, the motor is set/controlled to maintain the pressure of the system using the signal from the pressure sensor. Upon completion of the coarse pressure regulation mode, the motor speed ramping is stopped (i.e., the speed is maintained at a base speed) and any further changes to the base motor speed resulting from the coarse mode may be instead implemented with two controllers such as PID (proportional, integral, derivative) controllers. During the fine pressure regulation mode, the target pressure is compared with a qualified pressure estimate to generate a first error signal that is applied to the first controller (e.g., a PID controller) to produce, by summing the PID output of the PID controller with the base speed of the motor, a motor speed setting for control of the motor using a second controller (e.g., a PID controller). The qualified pressure estimate for the first PID controller is computed using regression. In this regard, samples from the pressure signal may be applied to a best fit algorithm (e.g., linear regression) to determine regression parameters (e.g., slope and intercept of a line) of the data from the pressure signal during an adsorption cycle. If the slope is positive, these parameters (e.g., slope and intercept rather than pressure samples from the pressure sensor) may then be applied with the particular time of the given adsorption phase of the pressure swing adsorption cycle to determine a peak value of the regression line from the linear regression. If the slope is negative, the intercept parameter may be taken as the peak value. The peak values from the regression information may be then applied to a running average buffer that maintains an average of the most recent peak values (e.g., six or more). The average peak value may then serve as the qualified pressure estimate for the controller. Versions of such processes are discussed in more detail in U.S. Provisional Patent Application No. 62/904,858, filed on 24 Sep. 2019, the entire disclosure of which is incorporated herein by reference.

Additionally, the controller of the POC may be configured to implement supply valve control to regulate bolus size (volume) in the system, which may optionally be implemented without use of a flow rate sensor of the POC. For example, the POC may be equipped with a pressure sensor, such as the pressure sensor 107 in the accumulator downstream of the sieve beds, and regulate bolus size, generated by the POC, as a function of pressure. Such regulation of bolus size may be a function of accumulator pressure.

7. Control Panel

Control panel 600 serves as an interface between a user and controller 400 to allow the user to initiate predetermined operation modes of the oxygen concentrator 100 and to monitor the status of the system. FIG. 1N depicts an implementation of control panel 600. Charging input port 605, for charging the internal power supply 180, may be disposed in control panel 600.

In some implementations, control panel 600 may include buttons to activate various operation modes for the oxygen concentrator 100. For example, control panel may include power button 610, flow rate setting buttons 620 to 626, active mode button 630, sleep mode button 635, altitude button 640, and a battery check button 650. In some implementations, one or more of the buttons may have a respective LED that may illuminate when the respective button is pressed, and may power off when the respective button is pressed again.

Power button 610 may power the system on or off. If the power button is activated to turn the system off, controller 400 may initiate a shutdown sequence to place the system in a shutdown state (e.g., a state in which both canisters are pressurized).

Flow rate setting buttons 620, 622, 624, and 626 allow a flow rate of oxygen enriched air to be selected (e.g., 0.2 LPM by button 620, 0.4 LPM by button 622, 0.6 LPM by button 624, and 0.8 LPM by button 626). In other implementations, the number of flow rate settings may be increased or decreased. After a flow setting is selected, oxygen concentrator 100 will then control operations to achieve production of the oxygen enriched air according to the selected flow rate setting.

Altitude button 640 may be activated when a user is going to be in a location at a higher elevation than the oxygen concentrator 100 is regularly used by the user.

Battery check button 650 initiates a battery check routine in the oxygen concentrator 100 that results in a relative battery power remaining LED 655 being illuminated on control panel 600.

A user may have a low breathing rate or depth if relatively inactive (e.g., asleep, sitting, etc.) as estimated by comparing the detected breathing rate or depth to a threshold. The user may have a high breathing rate or depth if relatively active (e.g., walking, exercising, etc.). An active/sleep mode may be estimated automatically and/or the user may manually indicate active mode or sleep mode by pressing button 630 for active mode or button 635 for sleep mode.

8. Methods of Operation

The methods of operating and monitoring oxygen concentrator 100 described below may be executed by the one or more processors, such as the one or more processors 410 of the controller 400, configured by program instructions, such as including, as previously described, the one or more functions and/or associated data corresponding thereto, stored in a memory such as the memory 420 of oxygen concentrator 100. Alternatively, some or all of the steps of the described methods may be similarly executed by one or more processors of an external computing device, such as the server 460, forming part of the connected respiratory therapy system 450, as described above. In this latter implementation, the processors 410 may be configured by program instructions stored in the memory 420 of oxygen concentrator 100 to transmit to the external computing device the measurements and parameters necessary for the performance of those steps that are to be carried out at the external computing device.

In order to minimize weight, size, and power consumption, oxygen concentrator 100 may deliver oxygen enriched air to the user as series of pulses. In such a pulsed oxygen delivery (POD) or demand mode of operation, controller 400 may regulate the size of the one or more released pulses or boluses to achieve delivery of the oxygen enriched air according to the selected flow rate setting. In order to maximize the effect of the delivered oxygen enriched air, controller 400 may be further programmed to synchronize the release of each bolus with the user's inhalations. Releasing a bolus of oxygen enriched air to the user as the user inhales may prevent wastage of oxygen by not releasing oxygen, for example, when the user is exhaling. The flow rate settings on control panel 600 may correspond to minute volumes (bolus volume multiplied by breathing rate per minute) of delivered oxygen (e.g., 0.2 LPM, 0.4 LPM, 0.6 LPM, 0.8 LPM, 1.1 LPM).

Oxygen enriched air produced by oxygen concentrator 100 is stored in accumulator 106 and, in a POD mode of operation, released to the user as the user inhales. The amount of oxygen enriched air provided by oxygen concentrator 100 is controlled, in part, by supply valve 160. In an implementation, supply valve 160 is opened for a sufficient amount of time to provide the appropriate amount of oxygen enriched air, as estimated by controller 400, to the user. In order to minimize the wastage of oxygen, the oxygen enriched air may be provided as a bolus soon after the onset of a user's inhalation is detected. For example, the bolus of oxygen enriched air may be provided in the first few milliseconds of a user's inhalation.

In some implementations, pressure sensor 194 may be used to determine the onset of inhalation by the user. For example, the user's inhalation may be detected by using pressure sensor 194. In use, delivery conduit 192 is coupled to the user's nose and/or mouth through nasal airway delivery device 196 and/or mouthpiece 198. The pressure in delivery conduit 192 is therefore representative of the user's airway pressure. At the onset of an inhalation, the user begins to draw air into their body through the nose and/or mouth. As the air is drawn in, a negative pressure is generated at the end of delivery conduit 192, due, in part, to the venturi action of the air being drawn across the end of delivery conduit 192. Controller 400 analyzes the pressure signal from pressure sensor 194 to detect a drop in pressure indicating the onset of inhalation. Upon detection of the onset of inhalation, supply valve 160 is opened to release a bolus of oxygen enriched air from accumulator 106.

In some implementations, pressure sensor 194 may be used to determine the onset of exhalation by the user. A positive change or rise in pressure in delivery conduit 192 indicates an exhalation by the user. Controller 400 may analyze the pressure signal from pressure sensor 194 to detect a rise in pressure indicating the onset of exhalation. In some implementations, when a positive pressure change is sensed, supply valve 160 is closed until the next onset of inhalation is detected. In other implementations, when a positive pressure change is sensed, supply valve 160 may be closed after a predetermined interval known as the bolus duration.

By measuring the intervals between adjacent onsets of inhalation, the user's breathing rate may be estimated. By measuring the intervals between onsets of inhalation and the subsequent onsets of exhalation, the user's inspiratory time may be estimated. In some implementations, the user's breathing rate and/or inspiratory time may be used to adjust the bolus duration. In some implementations, if the user's activity level (e.g., the user's breathing rate) exceeds a predetermined threshold, controller 400 may implement an alarm (e.g., visual and/or audio) to warn the user that the current breathing rate is exceeding the delivery capacity of oxygen concentrator 100. For example, the threshold may be set at 40 breaths per minute (BPM).

In other implementations, pressure sensor 194 may be positioned at different locations. For example, pressure sensor 194 may be located in a sensing conduit that is in pneumatic communication with the user's airway, but separate from delivery conduit 192. In such implementations, the pressure signal from pressure sensor 194 is still representative of the user's airway pressure. As another example, pressure sensor 194 may be placed in nasal cannula airway delivery device 196. In such implementations, a signal from pressure sensor 194 may be provided to controller 400 via one or more electrical conduits or one or more wireless transmitters, receivers, and/or transceivers. In some implementations, the sensitivity of pressure sensor 194 may be affected by the physical distance of pressure sensor 194 from the user, especially if pressure sensor 194 is located in oxygen concentrator 100 and the pressure difference is detected through delivery conduit 192 coupling the oxygen concentrator 100 to the user. Placement of pressure sensor 194 in nasal cannula airway delivery device 196 may improve its sensitivity.

FIG. 2 is a block diagram illustrating an adaptive triggering system 700 having an adjustment module 710, a threshold module 720, a trigger module 730, and a monitoring module 740 that may be implemented by oxygen concentrator 100 during a POD mode of operation. The various modules of system 700 may be implemented as processing components of system 700 or otherwise encoded as program instructions stored in memory 420 and executed by controller 400. While the functionality of the various modules may be as set out below, in other implementations, the functionality may be partitioned differently between the modules.

Adjustment module 710 may be configured to receive, for example, a measured pressure signal (e.g., a signal generated by pressure sensor 194), a valve control signal (e.g., a signal generated by controller 400 to control supply valve 160), and/or a measured temperature signal (e.g., a signal generated by a temperature sensor in oxygen concentrator 100). Adjustment module 710 may be configured to adjust the measured pressure signal so that it more accurately represents the user's airway pressure. For example, adjustment module 710 may use the valve control signal to remove the pressure pulse(s) or pressure effect(s) that is/are contained in the measured pressure signal as a consequence of each release of a bolus of oxygen enriched air. As another example, adjustment module 710 may use the measured temperature signal to compensate for variations in temperature by removing any offset drift (e.g., thermal or other) in the measured pressure signal that may be caused by those variations (e.g., pressure sensor 194 may be temperature sensitive). As yet another example, adjustment module 710 may perform noise reduction filtering on the measured pressure signal. The output of adjustment module 710 is an adjusted pressure signal as a function of time.

Threshold module 720 may be configured to monitor the adjusted pressure signal from adjustment module 710 and repeatedly determine an appropriate trigger threshold as a function of time. Threshold module 720 may have an activity estimation sub-module configured to generate an activity signal from the adjusted pressure signal. In some implementations, the activity signal may correspond with a breathing parameter, such as a breathing rate of the user. In some implementations, the activity signal may be indicative of other types of activity. For example, a filter (e.g., a high-pass filter, such as a second-order Butterworth high-pass filter) with an appropriate cutoff frequency (e.g., 10 Hz) may be used to generate an activity signal that is indicative of non-respiratory activity.

Threshold module 720 may also have a threshold update sub-module configured to adjust the trigger threshold based on the activity signal from the activity estimation sub-module. For example, the threshold update sub-module may increase the magnitude of the trigger threshold when the activity signal indicates an increase in the user's activity. Similarly, the threshold update sub-module may decrease the magnitude of the trigger threshold when the activity signal indicates a decrease in the user's activity. These adjustments may help compensate for increased noise in the adjusted pressure signal during periods of increased activity of the user.

In some implementations, the threshold update sub-module may analyze fixed-length windows (e.g., time periods of 5 to 15 seconds) of the activity signal. In other implementations, the threshold update sub-module may analyze adjustable-length windows of the activity signal. In such implementations, threshold module 720 may also have a window adjustment sub-module that is configured to adjust the length of the window used by the threshold update sub-module as a function of the activity signal, the adjusted pressure signal, and/or the trigger threshold. For example, the window adjustment sub-module may temporarily shorten the length of the window to allow the trigger threshold to make a quick recovery from a brief isolated episode of increased noise (e.g., from a cough or a cannula bump, whereby the cannula bump may be an agitation caused by physical contact with a part of the cannula). In such implementations, the window adjustment sub-module may adjust the length of the window based on the amount of time that the trigger threshold exceeded a recent moving average of the trigger threshold.

Trigger module 730 may be configured to apply the trigger threshold from threshold module 720 to the adjusted pressure signal from adjustment module 710 to generate a trigger signal (e.g., a digital Boolean signal or a proportional control signal). The trigger signal may be used to synchronize the release of a bolus of oxygen enriched air with the user's inhalation. For example, the trigger signal may be provided to supply valve 160. In some implementations, trigger module 730 may compare the adjusted pressure signal to the trigger threshold to identify an onset of inhalation. In such implementations, trigger module 730 may detect an onset of inhalation when the magnitude of the adjusted pressure signal is greater than the magnitude of the trigger threshold. In some implementations, trigger module 730 may also compare the time since the previously detected onset of inhalation to a blackout period. In such implementations, trigger module 730 may only detect an onset of inhalation when the time since the previously detected onset of inhalation is greater than the blackout period. In some implementations, trigger module 730 may also only detect an onset of inhalation when an onset of expiration has been detected after the previously detected onset of inhalation.

Monitoring module 740 may be configured to calculate one or more breathing parameters of the user (e.g., the user's breathing rate or inspiratory time) based on the adjusted pressure signal from adjustment module 710 and the trigger signal from trigger module 730. For example, monitoring module 740 may estimate the user's current breathing rate as the reciprocal of either a single recent breath duration or a moving average of two or more recent breath durations. A breath duration may be estimated as the length of time between successive detections of onsets of inhalation. As another example, monitoring module 740 may estimate the user's inspiratory time as the time for which the adjusted pressure has continuously remained below a predetermined threshold (e.g., zero). One or more of the breathing parameters calculated by monitoring module 740 may be provided to trigger module 730. In some implementations, trigger module 730 may adjust the length of the blackout period based on these breathing parameters. For example, trigger module 730 may decrease the length of the blackout period in response to an increase in the user's breathing rate. Similarly, trigger module 730 may increase the length of the blackout period in response to a decrease in the user's breathing rate. One or more of the breathing parameters calculated by monitoring module 740 may also be provided to one or more modules external to system 700 (e.g., a bolus adjustment module or a user data reporting module).

Further details regarding adaptive triggering systems may be found, for example, in International Patent Application No. PCT/AU2019/050302, entitled “Methods and Apparatus for Treating a Respiratory Disorder,” which is incorporated herein by reference.

In some implementations, flow rate sensor 185 may be used to determine the onset of inhalation and/or exhalation by the user. For example, in much the same way that controller 400 may analyze a pressure signal from pressure sensor 194 to detect a drop in pressure indicating the onset of inhalation, controller 400 may analyze a flow signal from flow rate sensor 185 to detect a negative flow rate indicating the onset of inhalation. Similarly, controller 400 may also analyze the flow signal from flow rate sensor 185 to detect a positive flow rate indicating the onset of exhalation. Upon detection of the onset of inhalation, supply valve 160 may be opened to release a bolus of oxygen enriched air from accumulator 106. Similarly, upon detection of the onset of exhalation, supply valve 160 may be closed until the next onset of inhalation is detected.

In some implementations, flow rate sensor 185 may be used in combination with pressure sensor 194 to determine the onset of inhalation and/or exhalation by the user. In such implementations, adaptive triggering system 700 of FIG. 2 may, for example, be modified such that adjustment module 710 also receives a measured flow signal (e.g., a signal generated by flow rate sensor 185). Furthermore, in such implementations, adjustment module 710 may be configured to produce both an adjusted pressure signal and an adjusted flow signal. Moreover, in such implementations, threshold module 720, trigger module 730, and/or monitoring module 740 may be reconfigured to use both the adjusted pressure signal and the adjusted flow signal to perform the operations described above.

In some implementations, flow rate sensor 185 may be used without pressure sensor 194 to determine the onset of inhalation and/or exhalation by the user. In such implementations, adaptive triggering system 700 of FIG. 2 may, for example, be modified such that adjustment module 710 is reconfigured to receive a measured flow signal (e.g., a signal generated by flow rate sensor 185) instead of a measured pressure signal. Furthermore, in such implementations, adjustment module 710 may be reconfigured to produce an adjusted flow signal instead of an adjusted pressure signal. Moreover, in such implementations, threshold module 720, trigger module 730, and/or monitoring module 740 may be reconfigured to use the adjusted flow signal to perform the operations described above.

In some implementations, oxygen concentrator 100 may initiate an automatic delivery mode when the time since the previously detected onset of inhalation is greater than a predetermined threshold. During the automatic delivery mode, boluses of oxygen enriched air are automatically delivered to the user regardless, for example, of whether or not an onset of inhalation is detected. The automatic delivery mode helps to ensure that the user still receives the prescribed amount of oxygen enriched air. In some implementations, oxygen concentrator 100 may exit the automatic delivery mode and resume a POD mode of operation after a user breath is detected. In some implementations, oxygen concentrator 100 may exit the automatic delivery mode and resume a POD mode of operation after a predetermined time period (e.g., 45 seconds, 1 minute, 2 minutes, 3 minutes, etc.)

In some implementations, the predetermined threshold used to initiate the automatic delivery mode is a fixed value (e.g., a time period of 5 to 15 seconds). In other implementations, the predetermined threshold is repeatedly adjusted. For example, the predetermined threshold may be repeatedly adjusted based on a moving average of two or more recent breath durations. For example, the predetermined threshold may be repeatedly calculated as the product of a scaling constant (e.g., 1.25, 1.5, 2, 2.5, etc.) and the moving average of two or more recent breath durations. As another example, the predetermined threshold may be repeatedly calculated as the sum of a predetermined time period (e.g., 2 seconds, 3 seconds, 4 seconds, etc.) and the moving average of two or more recent breath durations.

In some implementations, the size and/or frequency of the boluses delivered during the automatic delivery mode is fixed. In other implementations, the size and/or frequency of the boluses is repeatedly adjusted. For example, the size and/or frequency of the boluses may be repeatedly adjusted based on a moving average of two or more recent breath durations. As another example, the size of the boluses automatically delivered to the user may correspond with the size of one or more boluses that were previously delivered to the user in response to one or more previously detected onsets of inhalation. Similarly, the rate at which the boluses are automatically delivered to the user may correspond with the rate at which one or more boluses were previously delivered to the user in response to one or more previously detected onsets of inhalation.

B. Movement Compensation

As the user of an oxygen concentrator moves, one or more components of the oxygen concentrator may also move. For example, as the user moves, a delivery conduit (e.g., delivery conduit 192) and/or an airway delivery device (e.g., nasal cannula airway delivery device 196 and/or mouthpiece 198) may also move. These movements may affect the measurements of one or more sensors in the oxygen concentrator (e.g., oxygen sensor 165, flow rate sensor 185, pressure sensor 194). For example, movement of delivery conduit 192 of oxygen concentrator 100 may create noise in the oxygen concentration, flow, and/or pressure signals of oxygen sensor 165, flow rate sensor 185, and/or pressure sensor 194, respectively. Therefore, in some implementations, one or more movement sensors may be included in the systems described above to compensate for noise created by movement of the user.

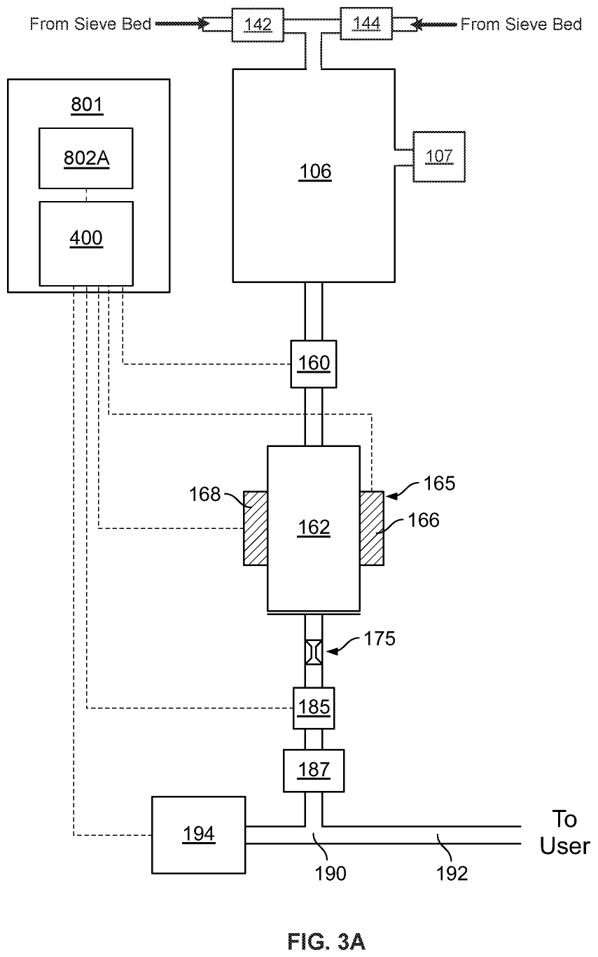

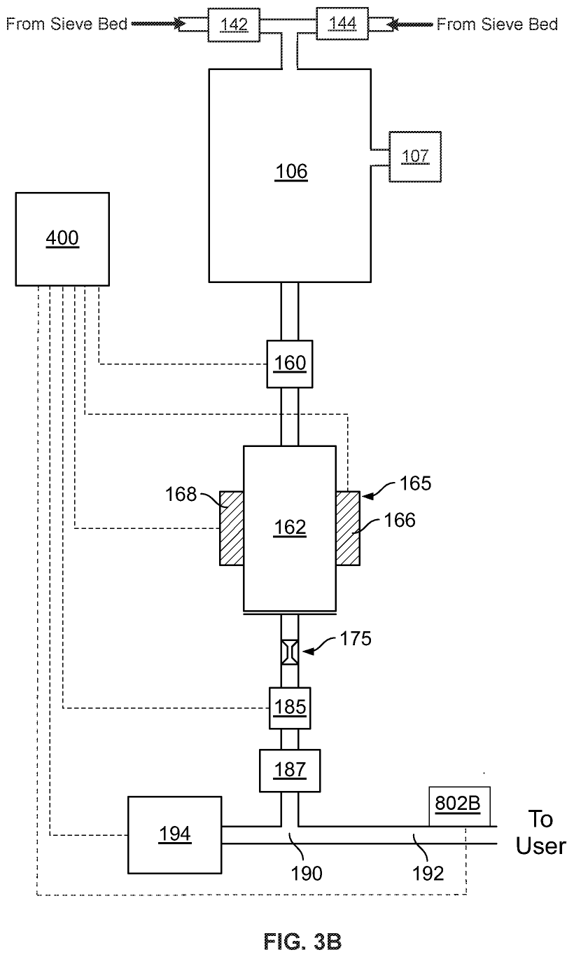

For example, as shown in FIGS. 3A and 3B, movement sensors may be included in oxygen concentrator 100. As shown in FIG. 3A, a movement sensor 802A is positioned on a controller board 801 with controller 400. As shown in FIG. 3B, a movement sensor 802B is positioned along delivery conduit 192. In some implementations, movement sensor 802B may be positioned closer to the user, and in other implementations, movement sensor 802B may be positioned closer to outer housing 170. In other implementations, movement sensors 802A and 802B may be positioned at different locations. For example, these sensors may be positioned anywhere inside oxygen concentrator 100, such as along a wall of outer housing 170 or on canister system 300. As another example, movement sensors 802A and 802B may be incorporated in a separate device from oxygen concentrator 100 that is, for example, carried or worn by a user. In such implementations, the separate device may, for example, be a personal cellular device or a wristwatch. In some implementations, a plurality of movement sensors may be included in oxygen concentrator 100. For example, movement sensors 802A and 802B may both be included in oxygen concentrator 100.

A variety of different movement sensors, such as accelerometers, gyroscopes, tilt switches, strain gauges, barometers, or altimeters, can be used with the present technology. For example, in some implementations, movement sensors 802A and/or 802B may be accelerometers configured to measure acceleration in one or more directions (e.g., a 1-axis accelerometer, a 2-axis accelerometer, or a 3-axis accelerometer). As another example, in some implementations, movement sensor 802B may be a strain gauge configured to measure bending of one or more portions of delivery conduit 192. As yet another example, in some implementations, movement sensors 802A and/or 802B may be barometers and/or altimeters configured to measure changes in altitude caused by the user.

Data generated by movement sensors 802A and 802B is received by controller 400. In some implementations, movement sensors 802A and 802B may be communicatively coupled to controller 400 through one or more electrical conduits. In such implementations, movement sensors 802A and 802B may transmit the generated data using standard communications protocols, such as Inter-Integrated Circuit (I²C), Serial Peripheral Interface (SPI), Controller Area Network (CAN), Universal Asynchronous Reception and Transmission (UART), Ethernet, or Universal Serial Bus (USB), or custom communications protocols. In some implementations, movement sensors 802A and 802B may wirelessly transmit the generated data to controller 400 through one or more wireless transmitters, receivers, and/or transceivers. In such implementations, movement sensors 802A and 802B may wirelessly transmit the generated data using standard communications protocols, such as Bluetooth, WiFi, ZigBee, Z-Wave, NEC Infrared (IR), Code Division Multiple Access (CDMA), Global System for Mobile Communications (GSM), or Long-Term Evolution (LTE), or custom communications protocols.

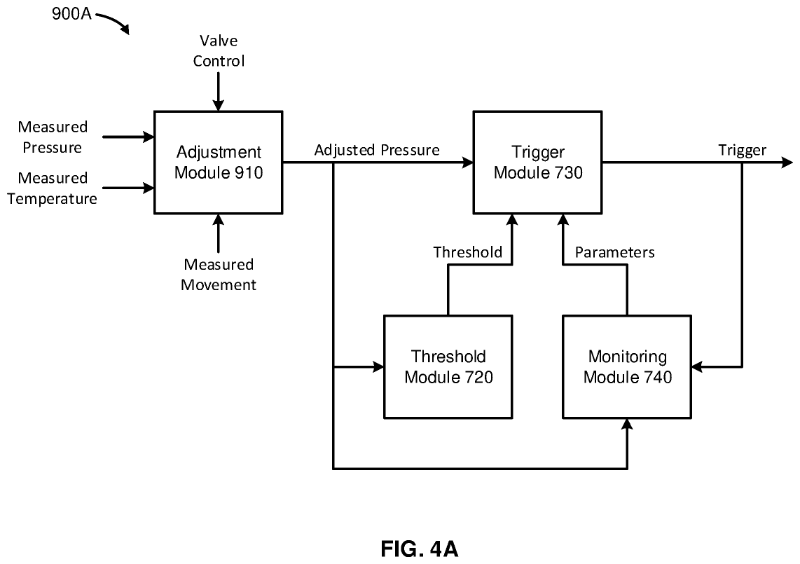

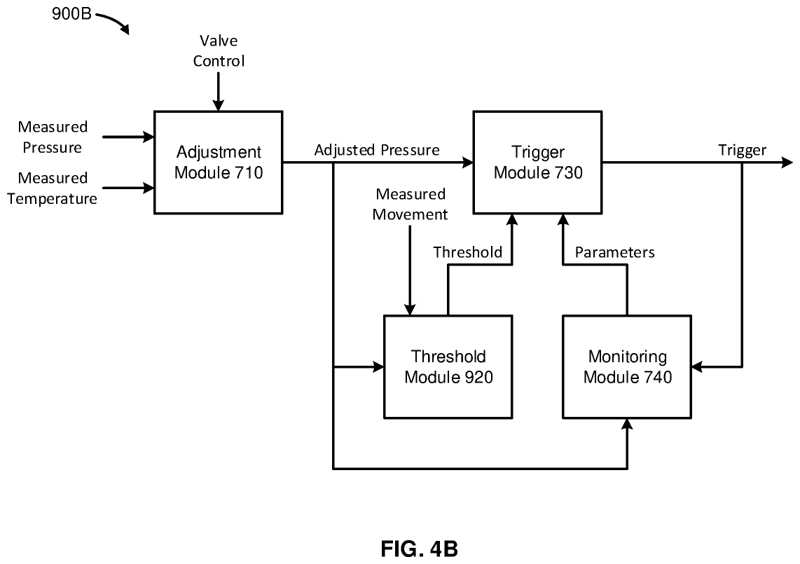

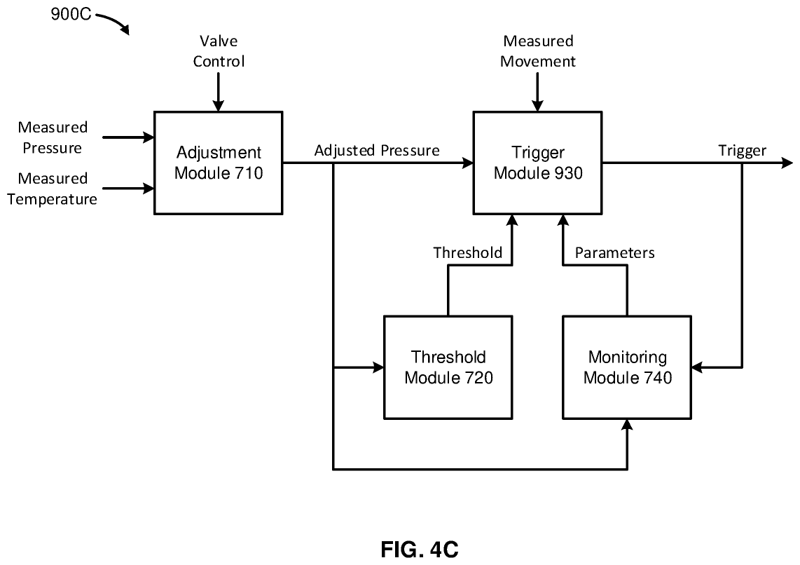

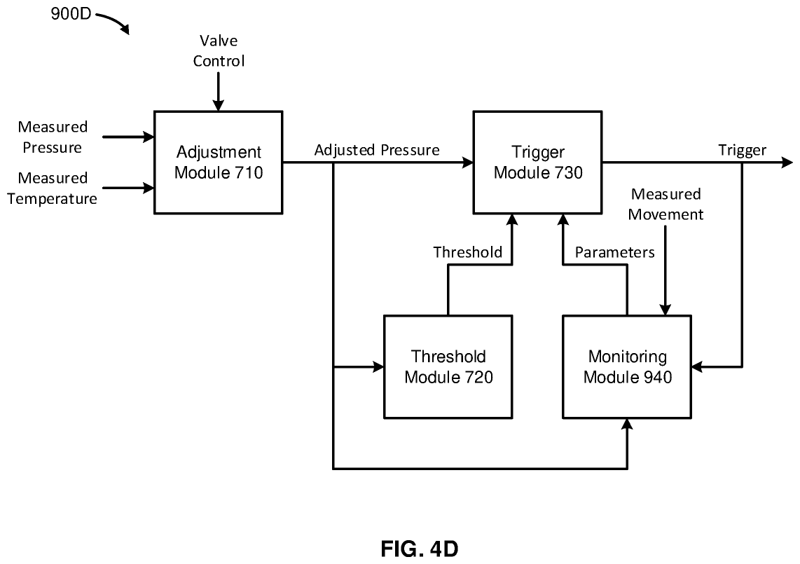

Controller 400 may use the data received from movement sensors 802A and 802B to compensate for noise created by movement of the user. For example, as shown in FIGS. 4A-4D, adaptive triggering system 700 of FIG. 2 may be modified to compensate for such noise. In each of the implementations of FIGS. 4A-4D, one of the modules of adaptive triggering system 700 (e.g., adjustment module 710, threshold module 720, trigger module 730, or monitoring module 740) has been replaced with a different module (e.g., adjustment module 910, threshold module 920, trigger module 930, or monitoring module 940). The remaining modules operate in the manner described above in relation to FIG. 2.

As shown in FIG. 4A, adjustment module 710 has been replaced with adjustment module 910 in adaptive triggering system 900A. Adjustment module 910 may be configured to receive, for example, a measured pressure signal (e.g., a signal generated by pressure sensor 194), a valve control signal (e.g., a signal generated by controller 400 to control supply valve 160), a measured temperature signal (e.g., a signal generated by a temperature sensor in oxygen concentrator 100), and/or a measured movement signal (e.g., a signal generated by movement sensor 802A or 802B). Much like adjustment module 710, adjustment module 910 may use the valve control signal, the measured temperature signal, and/or noise reduction filtering to adjust the measured pressure signal so that it more accurately represents the user's airway pressure. Additionally, adjustment module 910 may use the measured movement signal to compensate for noise created by movement of the user. For example, in implementations where adaptive triggering system 900A is used with the outlet systems of FIGS. 3A and/or 3B, adjustment module 910 may increase or decrease the measured pressure signal generated by pressure sensor 194 based on the measured movement signal generated by movement sensors 802A and/or 802B. For example, in implementations where movement sensors 802A and/or 802B are accelerometers, a measured direction of acceleration in relation to an orientation of pressure sensor 194 may indicate whether the measured pressure signal should be increased or decreased. As another example, in implementations where movement sensor 802B is a strain gauge, a measured bending of one or more portions of delivery conduit 192 may indicate whether the measured pressure signal should be increased or decreased. As yet another example, in implementations where movement sensors 802A and/or 802B are barometers and/or altimeters, a measured change in altitude may indicate whether the measured pressure signal should be increased or decreased.

As shown in FIG. 4B, threshold module 720 has been replaced with threshold module 920 in adaptive triggering system 900B. Much like threshold module 720, threshold module 920 may be configured to monitor the adjusted pressure signal from adjustment module 710 and repeatedly determine an appropriate trigger threshold as a function of time. Furthermore, threshold module 920 may have an activity estimation sub-module, a threshold update sub-module, and/or a window adjustment sub-module. However, the functionality of at least one of these sub-modules may be modified based on the measured movement signal.

Activity estimation sub-module may be configured to generate an activity signal based on the adjusted pressure signal and/or a measured movement signal (e.g., a signal generated by movement sensor 802A or 802B). For example, in some implementations, activity estimation sub-module may derive a breathing parameter from the adjusted pressure signal (e.g., a breathing rate of the user) and a movement parameter from the measured movement signal (e.g., a number of steps taken per unit of time by the user). Activity estimation sub-module may then combine the breathing parameter and the movement parameter to generate the activity signal. For example, the activity signal may be calculated as a weighted sum of the breathing parameter and the movement parameter. As another example, in some implementations, activity estimation sub-module may generate a non-respiratory signal from the adjusted pressure signal (e.g., using a high-pass filter with an appropriate cutoff frequency). Activity estimation sub-module may then scale the non-respiratory signal based on the measured movement signal. For example, when the measured movement signal indicates a larger amount of movement by the user, a larger scaling factor may be applied to the non-respiratory signal to generate the activity signal. Similarly, when the measured movement signal indicates a smaller amount of movement by the user, a smaller scaling factor may be applied to the non-respiratory signal to generate the activity signal.

Threshold update sub-module may be configured to adjust the trigger threshold based on the activity signal from the activity estimation sub-module and/or a measured movement signal (e.g., a signal generated by movement sensor 802A or 802B). For example, the threshold update sub-module may increase the magnitude of the trigger threshold when the activity signal reliably indicates an increase in the user's activity. Similarly, the threshold update sub-module may decrease the magnitude of the trigger threshold when the activity signal reliably indicates a decrease in the user's activity. The threshold update sub-module may use the measured movement signal to assess the reliability of the activity signal. For example, in implementations where adaptive triggering system 900B is used with the outlet systems of FIGS. 3A and/or 3B, movement of one or more components of oxygen concentrator 100 may affect the measurements of pressure sensor 194, which will in turn affect the activity signal. Therefore, when a magnitude and/or frequency of a movement signal generated by movement sensors 802A and/or 802B is greater than a predetermined threshold, the threshold update sub-module may, for example, temporarily ignore the activity signal and maintain the trigger threshold at its current value.

In some implementations, the threshold update sub-module may analyze fixed-length windows (e.g., time periods of 5 to 15 seconds) of the activity signal. In other implementations, the threshold update sub-module may analyze adjustable-length windows of the activity signal. In such implementations, threshold module 920 may have a window adjustment sub-module that is reconfigured to adjust the length of the window used by the threshold update sub-module as a function of the activity signal, the adjusted pressure signal, the trigger threshold, and/or a measured movement signal (e.g., a signal generated by movement sensor 802A or 802B). For example, the window adjustment sub-module may temporarily shorten the length of the window to allow the trigger threshold to make a quick recovery from a brief isolated episode of increased noise (e.g., from a cough or a cannula bump). In such implementations, the window adjustment sub-module may adjust the length of the window based on the amount of time that the trigger threshold exceeded a recent moving average of the trigger threshold. In some implementations, the window adjustment sub-module may be configured to analyze the measured movement signal to identify episodes of increased noise. For example, an episode of increased noise may be identified by the window adjustment sub-module when a magnitude and/or frequency of the measured movement signal is greater than a predetermined threshold.

As shown in FIG. 4C, trigger module 730 has been replaced with trigger module 930 in adaptive triggering system 900C. Much like trigger module 730, trigger module 930 may be configured to apply the trigger threshold from threshold module 720 to the adjusted pressure signal from adjustment module 710 to generate a trigger signal (e.g., a digital Boolean signal or a proportional control signal). The trigger signal may be used to synchronize the release of a bolus of oxygen enriched air with the user's inhalation. For example, the trigger signal may be provided to supply valve 160. In some implementations, trigger module 930 may compare the adjusted pressure signal to the trigger threshold to identify an onset of inhalation. In such implementations, trigger module 930 may detect an onset of inhalation when the magnitude of the adjusted pressure signal is greater than the magnitude of the trigger threshold. Much like trigger module 730, trigger module 930 may use blackout periods and/or detections of onsets of expiration to reduce the risk of falsely detecting onsets of inhalations. However, trigger module 930 may also use a measured movement signal (e.g., a signal generated by movement sensor 802A or 802B) to reduce the risk of falsely detecting onsets of inhalations. For example, trigger module 930 may verify an onset of inhalation when a magnitude and/or frequency of the measured movement signal is less than a predetermined threshold.

As shown in FIG. 4D, monitoring module 740 has been replaced with monitoring module 940 in adaptive triggering system 900D. Much like monitoring module 740, monitoring module 940 may be configured to calculate one or more breathing parameters of the user (e.g., the user's breathing rate or inspiratory time) based on the adjusted pressure signal from adjustment module 710 and the trigger signal from trigger module 730. However, monitoring module 940 may also be configured to calculate one or more movement parameters of the user (e.g., a number of steps taken per unit of time by the user) based on a measured movement signal (e.g., a signal generated by movement sensor 802A or 802B). Monitoring module 940 may also use the measured movement signal to increase the accuracy of the calculation of one or more breathing parameters. For example, monitoring module 940 may exclude one or more segments of the adjusted pressure signal from the calculation of a breathing parameter when a magnitude and/or frequency of one or more corresponding segments of the measured movement signal are greater than a predetermined threshold. Much like the breathing parameters calculated by monitoring module 740, the breathing and/or movement parameters calculated by monitoring module 940 may be provided to trigger module 730 and/or one or more modules external to system 900D (e.g., a bolus adjustment module or a user data reporting module).

In the implementations of FIGS. 4A-4D, only one of the modules of adaptive triggering system 700 (e.g., adjustment module 710, threshold module 720, trigger module 730, or monitoring module 740) was replaced with a different module (e.g., adjustment module 910, threshold module 920, trigger module 930, or monitoring module 940). However, in other implementations, multiple modules and/or sub-modules can be replaced. For example, two or more of adjustment module 910, threshold module 920, trigger module 930, and/or monitoring module 940 may be incorporated into an adaptive triggering system.

In the implementations of FIGS. 4A-4D, a measured pressure signal (e.g., a signal generated by pressure sensor 194) was used to determine the onset of inhalation and/or exhalation by the user. However, as explained above, in other implementations, a measured flow signal (e.g., a signal generated by flow rate sensor 185) may be used to determine the onset of inhalation and/or exhalation by the user. In such implementations, the measured flow signal may be used with or without a measured pressure signal (e.g., a signal generated by pressure sensor 194).

As noted above, in some implementations, oxygen concentrator 100 may initiate an automatic delivery mode when the time since the previously detected onset of inhalation is greater than a predetermined threshold. During the automatic delivery mode, boluses of oxygen enriched air are automatically delivered to the user regardless, for example, of whether or not an onset of inhalation is detected. In some implementations, the size and/or frequency of the boluses is repeatedly adjusted. For example, one or more breathing parameters calculated by monitoring module 940 may be used to adjust the size and/or frequency of the boluses. As another example, one or more separately calculated breathing parameters may be used to adjust the size and/or frequency of the boluses (e.g., a moving average of two or more recent breath durations). In such implementations, a measured movement signal (e.g., a signal generated by movement sensor 802A or 802B) can be used to increase the accuracy of these calculations. For example, one or more segments of a measured flow signal (e.g., a signal generated by flow rate sensor 185) and/or a measured pressure signal (e.g., a signal generated by pressure sensor 194) may be excluded from the calculation of the breathing parameters when a magnitude and/or frequency of one or more corresponding segments of the measured movement signal are greater than a predetermined threshold.

In some implementations, additional sensors may be incorporated into the systems and methods described above. For example, a measured heart rate signal generated by a heart rate monitor may be used in combination with a measured movement signal (e.g., a signal generated by movement sensor 802A or 802B) to compensate for noise created by movement of the user. In such implementations, the measured heart rate signal may be supplied to any of the modules described above. Increased heart rate may indicate increased movement by the user. Similarly, decreased heart rate may also indicate decreased movement by the user. As a result, adjustment module 910 may, for example, also use the measured heart rate signal to adjust the measured pressure. As another example, activity estimation sub-module of threshold module 920 may derive a heart rate parameter from the measured heart rate signal. Activity estimation sub-module may then combine the heart rate parameter with the breathing parameter and the movement parameter to generate the activity signal. As yet another example, the threshold update sub-module of threshold module 920 may use the measured heart rate signal to assess the reliability of the activity signal. As yet another example, the window adjustment sub-module of threshold module 920 may adjust the length of the window based on a magnitude and/or frequency of the measured heart rate signal. As yet another example, the trigger module 930 may verify an onset of inhalation based on a magnitude and/or frequency of the measured heart rate signal. As yet another example, the monitoring module 940 may exclude one or more segments of the adjusted pressure signal from the calculation of a breathing parameter based on a magnitude and/or frequency of the measured heart rate signal. As yet another example, the measured heart rate signal may be used to adjust the size and/or frequency of the boluses delivered during an automatic delivery mode.

D. Glossary

For the purposes of the present technology disclosure, in certain forms of the present technology, one or more of the following definitions may apply. In other forms of the present technology, alternative definitions may apply.

Air: In certain forms of the present technology, air may be taken to mean atmospheric air, consisting of 78% nitrogen (N₂), 21% oxygen (O₂), and 1% water vapour, carbon dioxide (CO₂), argon (Ar), and other trace gases.

Oxygen enriched air: Air with a concentration of oxygen greater than that of atmospheric air (21%), for example at least about 50% oxygen, at least about 60% oxygen, at least about 70% oxygen, at least about 80% oxygen, at least about 87% oxygen, at least about 90% oxygen, at least about 95% oxygen, at least about 98% oxygen, or at least about 99% oxygen. “Oxygen enriched air” is sometimes shortened to “oxygen.”

Medical Oxygen: Medical oxygen is defined as oxygen enriched air with an oxygen concentration of 80% or greater.

Ambient: In certain forms of the present technology, the term ambient will be taken to mean (i) external of the treatment system or patient, and (ii) immediately surrounding the treatment system or patient.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

Flow therapy: Respiratory therapy comprising the delivery of a flow of air to an entrance to the airways at a controlled flow rate referred to as the treatment flow rate that is typically positive throughout the patient's breathing cycle.

Patient: A person, whether or not they are suffering from a respiratory disorder.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar ˜0.001 atm). In this specification, unless otherwise stated, pressure is given in units of cmH₂O.

E. General Remarks

The term “coupled” as used herein means either a direct connection or an indirect connection (e.g., one or more intervening connections) between one or more objects or components. The phrase “connected” means a direct connection between objects or components such that the objects or components are connected directly to each other. As used herein the phrase “obtaining” a device means that the device is either purchased or constructed.

In the present disclosure, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative implementations of various aspects of the present technology may be apparent to those skilled in the art in view of this description. Accordingly, this description is to be construed as illustrative only and is for the purpose of teaching those skilled in the art the general manner of carrying out the technology. It is to be understood that the forms of the technology shown and described herein are to be taken as implementations. Elements and materials may be substituted for those illustrated and described herein, parts and processes may be reversed, and certain features of the technology may be utilized independently, all as would be apparent to one skilled in the art after having the benefit of this description of the technology. Changes may be made in the elements described herein without departing from the spirit and scope of the technology as described in the appended claims.

1. An oxygen concentration system comprising:
a pressure sensor configured to generate pressure signals, wherein the pressure sensor is pneumatically coupled to a delivery conduit for providing a user with oxygen enriched air;
a movement sensor configured to generate a movement signal; and
one or more processors communicatively coupled to the pressure sensor and the movement sensor, wherein the one or more processors are configured to:
adjust a trigger threshold based on an initial pressure signal obtained from the pressure sensor and the movement signal obtained from the movement sensor; and
compare the adjusted trigger threshold with a subsequent pressure signal obtained from the pressure sensor to determine when to provide the user with a bolus of oxygen enriched air through the conduit.

2. The system of claim 1, wherein the one or more processors are further configured to maintain the trigger threshold when a magnitude or a frequency of the movement signal is greater than a predetermined threshold.

3. The system of claim 1, wherein adjusting the trigger threshold based on the initial pressure signal and the movement signal comprises:
generating an activity signal;
increasing a magnitude of the trigger threshold when a window of the activity signal indicates an increase in the user's activity; and
decreasing a magnitude of the trigger threshold when the window of the activity signal indicates a decrease in the user's activity.

4. The system of claim 3, wherein generating the activity signal comprises:
deriving at least one breathing parameter from the initial pressure signal;
deriving at least one movement parameter from the movement signal; and
combining the at least one breathing parameter and the at least one movement parameter to generate the activity signal.

5. The system of claim 4, wherein the at least one breathing parameter is a breathing rate of the user, and wherein the at least one movement parameter is a number of steps taken per unit of time by the user.

6. The system of claim 3, wherein generating the activity signal comprises:
generating a non-respiratory signal from the initial pressure signal; and
scaling the non-respiratory signal based on the movement signal to generate the activity signal.

7. The system of claim 6, wherein a filter is applied to the initial pressure signal to generate the non-respiratory signal.

8. The system of claim 3, wherein a length of the window is fixed.

9. The system of claim 3, wherein adjusting the trigger threshold based on the initial pressure signal and the movement signal further comprises adjusting a length of the window based on the movement signal.

10. The system of claim 9, wherein adjusting the length of the window based on the movement signal comprises shortening the length of the window when a magnitude or a frequency of the movement signal is greater than a predetermined threshold.

11. The system of claim 1, wherein the movement sensor comprises an accelerometer coupled to the delivery conduit.

12. The system of claim 1, wherein the movement sensor comprises a strain gauge coupled to the delivery conduit.

13. The system of claim 1 further comprising:
a compression system configured to generate a pressurized stream of ambient air; and
a canister system comprising a canister containing a gas separation adsorbent, wherein the gas separation adsorbent is configured to separate at least some nitrogen from the pressurized stream of ambient air to produce oxygen enriched air.

14. An oxygen concentration system comprising:
a pressure sensor configured to generate pressure signals, wherein the pressure sensor is pneumatically coupled to a delivery conduit for providing a user with oxygen enriched air;
a movement sensor configured to generate a movement signal; and
one or more processors communicatively coupled to the pressure sensor and the movement sensor, wherein the one or more processors are configured to:
adjust a pressure signal obtained from the pressure sensor based on the movement signal obtained from the movement sensor; and
compare a trigger threshold with the adjusted pressure signal to determine when to provide the user with a bolus of oxygen enriched air through the conduit.

15. The system of claim 14, wherein the movement sensor comprises an accelerometer coupled to the delivery conduit.

16. The system of claim 15, wherein adjusting the pressure signal based on the movement signal comprises analyzing a direction of acceleration derived from the movement signal in relation to an orientation of the pressure sensor.

17. The system of claim 14, wherein the movement sensor comprises a strain gauge coupled to the delivery conduit, and wherein adjusting the pressure signal based on the movement signal comprises analyzing a measured bending of one or more portions of the delivery conduit.

18. The system of claim 14, wherein the one or more processors are further configured to adjust the trigger threshold based on the movement signal before the trigger threshold is compared with the adjusted pressure signal.

19. An oxygen concentration system comprising:
a pressure sensor configured to generate pressure signals, wherein the pressure sensor is pneumatically coupled to a delivery conduit for providing a user with oxygen enriched air;
a movement sensor configured to generate a movement signal; and
one or more processors communicatively coupled to the pressure sensor and the movement sensor, wherein the one or more processors are configured to:
detect a potential onset of inhalation by comparing a trigger threshold with a pressure signal obtained from the pressure sensor;
determine whether to verify the potential onset of inhalation based on the movement signal obtained from the movement sensor; and
provide the user with a bolus of oxygen enriched air through the conduit if the potential onset of inhalation is verified.

20. The system of claim 19, wherein determining whether to verify the potential onset of inhalation based on the movement signal comprises comparing a magnitude of the movement signal to a predetermined threshold.

21. The system of claim 20, wherein the potential onset of inhalation is verified if the magnitude of the movement signal is less than the predetermined threshold.

22. The system of claim 19, wherein determining whether to verify the potential onset of inhalation based on the movement signal comprises comparing a frequency of the movement signal to a predetermined threshold.

23. The system of claim 22, wherein the potential onset of inhalation is verified if the frequency of the movement signal is less than the predetermined threshold.

24. The system of claim 19, wherein the movement sensor comprises an accelerometer coupled to the delivery conduit.

25. A method of generating a trigger signal for controlling release of a bolus of oxygen enriched gas from an oxygen concentrator, the method comprising:
calculating a trigger threshold from an initial pressure signal representing an airway pressure of a user and a movement signal;
comparing a subsequent pressure signal representing the airway pressure of the user with the trigger threshold; and
generating, based on the comparison, the trigger signal for controlling release of the bolus.

26. A method of generating a trigger signal for controlling release of a bolus of oxygen enriched gas from an oxygen concentrator, the method comprising:
adjusting a pressure signal representing an airway pressure of a user based on a movement signal;
comparing the adjusted pressure signal with a trigger threshold; and
generating, based on the comparison, the trigger signal for controlling release of the bolus.

27. A method of generating a trigger signal for controlling release of a bolus of oxygen enriched gas from an oxygen concentrator, the method comprising:
comparing a pressure signal with a trigger threshold to detect a potential onset of inhalation;
determining whether to verify the potential onset of inhalation based on a movement signal; and
generating, based on the verifying, the trigger signal for controlling release of the bolus.
